(19) United States
(12) Patent Application Publication
(10) Pub. No.: US 2017/0165420 A1
(43) Pub. Date: Jun. 15, 2017

(54) SUBCUTANEOUS HYDRATION SYSTEM, METHOD, AND DEVICE

(71) Applicant: University Hospitals Case Medical Center, Cleveland, OH (US)

(72) Inventors: Johnie Rose, Cleveland, OH (US); Conor P. Delaney, Cleveland, OH (US)

(73) Assignee: University Hospitals Case Medical Center

(21) Appl. No.: 15/369,149

(22) Filed: Dec. 5, 2016

Related U.S. Application Data
(63) Continuation-in-part of application No. 14/211,006, filed on Mar. 14, 2014, now Pat. No. 9,511,195.
(60) Provisional application No. 61/793,200, filed on Mar. 15, 2013.

(51) Int. Cl. A61M 5/158; A61M 5/14

(52) U.S. Cl. CPC A61M 5/158; A61M 5/1411; A61M 5/1417; A61M 2005/1585; A61M 2205/0205; A61M 2005/1586; A61M 2005/1402; A61M 2005/1403

(57) ABSTRACT

Systems, methods, and devices for providing subcutaneous hydration are disclosed. A system for providing subcutaneous hydration may include an infusion bag including a hydration fluid, a tubing removably connected to the infusion bag, a squeezable bulb at least partially encompassing the tubing for at least one of flushing and priming the tubing, and a deployment device including an integrated needle which provides subcutaneous hydration through the needle. A deployment device for subcutaneous hydration includes a base having a needle aperture, a casing attached to the base, a firing mechanism within the casing for deploying a needle from the casing, and at least one wing attached to the base that includes an actuator that regulates the firing mechanism. A method for providing subcutaneous hydration may include steps necessary to operate the systems and devices for subcutaneous hydration.

### CROSS-REFERENCE TO RELATED APPLICATIONS

This application is a continuation-in-part of U.S. patent application Ser. No. 14/211,006, now U.S. Pat. No. 9,511,195, filed Mar. 14, 2014, which claims priority to U.S. Provisional Patent Application Ser. No. 61/793,200, filed on Mar. 15, 2013. The entirety of these applications are fully incorporated by reference herein.

### BACKGROUND

The present disclosure relates to systems, methods, and devices for providing subcutaneous hydration. Generally, the system may be used to provide subcutaneous hydration in a cost effective, integrated, and simple manner that is performable by non-clinically trained administrators. Methods to produce a deployment device and to provide subcutaneous hydration are also disclosed. The device may be an integrated unit for delivering subcutaneous hydration to patient in safe, cost-effective, durable, and/or less risky manner.

Generally practiced methods for rehydrating patients who suffer from severe dehydration include intravenous (IV) rehydration and oral rehydration therapy (ORT). IV rehydration provides a fast aqueous drip rate and is preferable when treating severely dehydrated patients. However, IVs also require individuals with a high level of skill for their set up and administration. By contrast, oral rehydration therapy does not require a high degree of skill to administer, yet also provides a slower rehydration effect and may not be feasible in certain patients.

Often, individuals in remote parts of the developing world either lack access to formal medical care where intravenous fluids can be administered, or face significant delay and transit times before obtaining medical care. Over 1.5 million children die annually due to diarrhea despite increasingly effective implementation of programs to increase access to oral rehydration solutions. Accordingly, the situation inevitably arises where oral rehydration therapy does not provide a sufficient rehydration rate for a specific patient, e.g. one who is overly dehydrated or suffering from emesis, while individuals with the skill necessary to set up IV rehydration on the patient are not readily available. In this case, an intermediary measure may be a more appropriate solution. This measure will not replace IV or ORT, but rather provide a practical bridge therapy which may help avert circulatory collapse.

One secondary or intermediary rehydration method involves subcutaneous, as opposed to intravenous, delivery of rehydrating fluids. Such devices should be easier to administer as no needle puncture of human veins is required. The set up time should also be much quicker, and the cost necessarily lower. Local clinically-trained personnel would not be required to administer this intermediary measure.

Current subcutaneous rehydration devices, e.g. a butterfly needle attached to a saline bag with an attached drip set, have a number of recognized limitations. First, they do not provide enough failsafe guidance for non-clinically trained administrators or self-administering patients. While subcutaneous injection does not puncture human veins, it may can cause unnecessary pain when the injection is performed improperly, e.g., using the wrong injection angle, depth, or injection site. Second, these devices may also not be integrated well enough for non-clinically trained administrators to provide facile treatment without confusion, delay, and/or impaired effectiveness.

It would be desirable to provide systems, methods, and devices for a self-contained subcutaneous hydration device which may be used by minimally-trained caregivers, which are the predominate type of caregivers in remote situations. It would also be desirable to provide durable, easy-to-use systems, methods, and devices for administering parenteral fluids using a subcutaneous temporizing measure when oral rehydration therapies are insufficient and IV fluids will be delayed or are unavailable. Such subcutaneous measure should provide sufficient guidance as to reduce the pain experienced by a patient when administered by non-clinically trained technicians. Such measures should also be well integrated to reduce confusion and loss of effectiveness, while also being cost effective for proliferate use in economically depressed regions, trauma or battlefield situations, and emergency and disaster relief.

### BRIEF DESCRIPTION

Disclosed in various embodiments are systems for providing subcutaneous hydration including an infusion bag having a hydration fluid; and tubing removably attached to the infusion bag which provides the hydration fluid to a deployment device attached to the tubing. The tubing is attached to a slidable lure within the deployment device at a connection point.

The lure may be removably attached to a needle operative to deliver the hydration fluid to a subcutaneous region of a patient. An adhesive base can be placed between the skin and the deployment device. The adhesive base may include an emulsion of at least one anesthetic such as prilocaine or lidocaine. In particular embodiments, the adhesive base includes at least one of polypropylene and tegaderm.

The deployment device may comprise an acrylic sheet.

Also disclosed in embodiments are deployment devices for subcutaneous hydration which include a wedge back attached to a wedge ramp which props the wedge back at a predefined angle, a slider track within the wedge ramp, a slider removably attached to the slider track and operative to translate along the wedge ramp, and a lure including a removably attached needle. The lure is at least partially encompassed by and directed by the movement of the slider into a dermal region injection site.

In particular embodiments, the wedge back and wedge ramp are made from acrylic and are attachable by adhesive. The predefined angle can be between 10 and 30 degrees. The needle is generally directed by the slider into a subcutaneous region and is operative to deliver hydration fluid to the subcutaneous region.

In other embodiments, the deployment device further includes a hole for at least one of inserting and translating the lure through the wedge back.

Also disclosed in several embodiments, are methods for forming a low-cost acrylic deployment device that include: forming first, second, and third portions of acrylic sheet from acrylic sheet stock, generating a slider track in the first sheet portion, cutting a cube out of acrylic sheet stock to form a slider, cutting at least two rectangular sections from the slider for interfacing the slider with the slider track; cutting a hollow cylinder out of the slider for receiving a lure, and forming a needle carriage from the first, second, and third acrylic sheet portions.

The needle carriage can be formed by gluing the acrylic sheet portions together. An angle of approximately 30 degrees may be maintained between acrylic sheet portions forming the smallest angle in the needle carriage.

Additionally disclosed herein are different embodiments of a system for providing subcutaneous hydration that include: an infusion bag including a hydration fluid, a tubing removably connected to the infusion bag, a squeezable bulb at least partially encompassing the tubing for at least one of flushing and priming the tubing, and a deployment device including an integrated needle. The deployment device is connected to the tubing and is operative to provide subcutaneous hydration through the needle.

The squeezable bulb may include a one-way valve. The tubing can be pre-connected to the infusion bag.

The present disclosure also relates to deployment devices for subcutaneous hydration that include: a base including a needle aperture, a spring-loaded barrel attached to the base and including a needle, and a firing mechanism within the barrel for deploying the needle from the barrel. The firing mechanism is operative to project the needle through the aperture only when a tent of skin has been raised below the aperture for injection.

The deployment device may further include at least one actuator attached to the barrel for regulating a needle slot. The deployment device may further include at least one wing attached to the base for engaging the at least one actuator. The at least one wing can engage the at least one actuator by gripping or pinching the at least one wing against the barrel.

The base may include at least one of an antimicrobial material and a waterproof adhesive. The barrel may at least partially encompass a tubing operative to provide hydration fluid to the needle.

The deployment device may further include a casing attached to the base which at least partially encompasses the barrel. The casing can include a slider track for retracting the needle within the spring-loaded barrel. The needle aperture may be oblong to accommodate needle movement while the needle is at least partially stabilized by the barrel.

The deployment device may further include an adhesive located on the base (530). The adhesive can be impregnated with an emulsion of at least one of prilocaine and lidocaine local anesthetic.

In still other embodiments disclosed herein are methods for providing subcutaneous hydration that include: hanging an infusion bag containing infusion liquid above a patient, penetrating the infusion bag by sliding a pre-connected tube towards the bag and twisting to lock, squeezing a bulb encompassing the tube to prime the tube, and pinching wings together on a deployment device until a click is felt, wherein a needle is automatically deployed and subcutaneous hydration is provided to the patient.

The wings are slowly released to allow them to return to a nearly-flat position. A slider can be translated until locked in order to withdraw the needle from the patient.

The present disclosure also relates to deployment devices for subcutaneous hydration that include: a base including a needle aperture; a casing attached to the base; a firing mechanism within the casing for deploying the needle from the casing/through the needle aperture; at least one wing attached to the base which has an actuator operative to regulate the firing mechanism and deployment of the needle. The firing mechanism projects/deploys the needle through the aperture when the wing is moved. Desirably, the wing is pushed downwards to cause deployment of the needle. Also desirably, the movement of the wing causes a tent of skin to be pulled up, with the needle being deployed into the tent of skin. In addition, the aperture desirably accommodates needle movement while the needle is at least partially stabilized by the device.

In particular embodiments, the firing mechanism includes: a slider with handles that travel through a slider track within the casing; a stop wall that engages the actuator; and the needle. The stop wall can be part of a retaining ring that is adapted to fix the needle in place on the slider, or can be an integral part of the slider itself. The slider may include a hollow passage, such that fluid can pass from a rear portion of the slider through the slider to the needle.

In some embodiments, the firing mechanism includes a spring between the casing and a rear portion of the slider.

The casing includes a slider track on two opposite sides, the slider track being angled relative to the base. The casing can further include at least one recessed notch located below the slider track for maintaining the firing mechanism in a retracted position.

The casing and the base may define a barrel. The device can further comprise an end cap that engages a rear wall of the casing to enclose the barrel. Generally, the firing mechanism is inserted into the barrel and is completely separable from the casing and the base. The end cap may further include projecting walls that define a rear end of a slider track.

In various embodiments, the base includes knuckles, and the wing includes knuckles, such that the wing and the base are hinged together.

The wing may comprise a frame, with the actuator extending transversely to the frame. The wing may also include a flat surface.

The needle may be deployed from the base at an angle. The angle may be from about 10 degrees to about 30 degrees.

In still other embodiments disclosed herein are methods for providing subcutaneous hydration that include providing a deployment device; hanging an infusion bag at a level above a patient's head; cleaning an area of skin of the patient; penetrating the infusion bag by sliding a pre-connected tube towards the bag and twisting to lock; squeezing a one-way valve bulb to prime the tube; removing a backing material from a base of the deployment device and applying the base flat to the skin of the patient; pushing at least one wing of the deployment device downward toward the skin of the patient until a needle is automatically deployed. A slider can be translated until locked in order to withdraw the needle from the patient.

These and other non-limiting aspects of the present disclosure are further discussed herein.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is an overview photograph of a system for providing subcutaneous hydration.

FIG. 2 is a perspective drawing of a deployment device for subcutaneous hydration according to a first exemplary embodiment.

FIG. 3 is a perspective drawing of a lure from the deployment device according to FIG. 2.

FIG. 4 is a technical diagram of the deployment device according to FIG. 2 which provides demonstrative dimensional features.

FIG. 5 is a drawing of the interface between a wedge back and a wedge ramp from the deployment device according to FIG. 2.

FIG. 6 is a drawing of the interface between a slider and a lure of the deployment device according to FIG. 2.

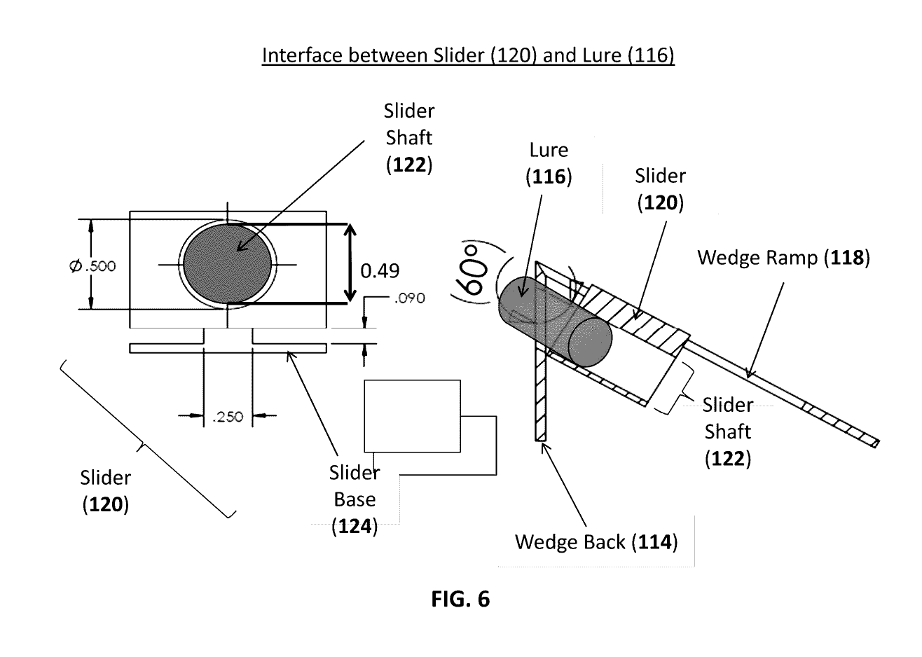

FIG. 7 is a drawing of the interface between the wedge back and the lure of the deployment device according to FIG. 2.

FIG. 8 is a flow chart of the potential hazards presented by subcutaneous deployment devices.

FIG. 9 is a drawing of the deployment device according to FIG. 2 provided with additional polypropylene sheet and tegaderm adhesive.

FIG. 10 is a drawing of (1) the interface between the wedge back, wedge ramp, and polypropylene sheet, (2) the interface between the polypropylene sheet and the tegaderm adhesive, and (3) the interface between the tegaderm adhesive and human skin or dermis, all interfaces from the deployment device according to FIG. 2.

FIG. 11 is a drawing of the interface between a subcutaneous layer of human skin and a needle of the deployment device according to FIG. 2.

FIG. 12 is a drawing illustrating the penetration of the needle from the deployment device according to FIG. 2 into the subcutaneous region.

FIG. 13 is a photograph and drawing of a deployment device for subcutaneous hydration according to a second exemplary embodiment.

FIG. 14 is a technical diagram of the deployment device according to FIG. 13 which provides demonstrative dimensional features.

FIG. 15 is a perspective drawing of a deployment device for subcutaneous hydration according to a third exemplary embodiment of the invention.

FIG. 16 is a technical diagram of the wedge back, side, and ramp from the deployment device according to FIG. 15 with demonstrative dimensional features.

FIG. 17 is a perspective drawing of the wedge back, side, and ramp from the deployment device according to FIG. 15 with a slider track prominently displayed.

FIG. 18 is a perspective drawing of the slider of the deployment device according to FIG. 15.

FIG. 19 is a technical diagram of the slider for the deployment device according to claim 15 with demonstrative dimensional figures.

FIG. 20 is two perspective drawings of the deployment device according to FIG. 15 in showing a start and finish position.

FIG. 21 is a manufacture process chart illustrating the different transformations an acrylic sheet undergoes when forming a slider for the deployment device according to FIG. 15.

FIG. 22 is a flow chart of a method for forming the deployment device according to FIG. 15.

FIG. 23 is an overview drawing of a system for providing subcutaneous hydration according to a second exemplary embodiment.

FIG. 24A is a bottom cutaway view, FIG. 24B is a rear cutaway view, and FIG. 24C is a top cutaway view drawing of an undeployed deployment device according to a third exemplary embodiment for use in the system of FIG. 23.

FIG. 25A is a bottom view, and FIG. 25B is a rear view of a deployed state of the deployment device according to FIG. 24A, FIG. 24B, and FIG. 24C.

FIG. 26 is a drawing of the firing mechanism (under an undeployed state) of the deployment device according to FIG. 24.

FIG. 27 is a 3D Model of a deployment device according to a fourth exemplary embodiment in a perspective view.

FIG. 28 is a bottom cutaway view of the 3D model of the deployment device according to FIG. 27. The needle appears to be deployed as it sticks out of the base.

FIG. 29 is a close-up cutaway view of the base of the 3D model of the deployment device according to FIG. 27 showing an oblong needle aperture.

FIG. 30 is a conceptual drawing showing the potential movement of the needle within the oblong needle aperture space in the deployment device according to FIG. 27.

FIG. 31 is a top cutaway view of the 3D model of the deployment device according to FIG. 27 prominently displaying a slider track for disengaging the needle after deployment.

FIG. 32 is a method for providing subcutaneous hydration according to an exemplary embodiment.

FIG. 33 is an exterior perspective view of a deployment device according to a fifth exemplary embodiment in a perspective view.

FIG. 34 is a perspective view of the interior of the deployment device according to FIG. 33.

FIG. 35 is an exploded view of the firing mechanism of the deployment device according to FIG. 33.

FIG. 36 is an exploded view showing the casing, the base, and the wings of the deployment device according to FIG. 33.

FIG. 37 is an exterior perspective view showing the rear of the deployment device according to FIG. 33. An end cap is attached.

FIG. 38 is an exterior rear perspective view of the casing of the deployment device according to FIG. 33, showing an interior view of the casing.

FIG. 39 is a perspective view of one wing of the deployment device of FIG. 33.

FIG. 40 is a perspective view of the base of the deployment device of FIG. 33.

FIG. 41 is a bottom view of the deployment device according to FIG. 33.

FIG. 42 is a method for providing subcutaneous hydration according to another exemplary embodiment.

### DETAILED DESCRIPTION

A more complete understanding of the processes and apparatuses disclosed herein can be obtained by reference to the accompanying drawings. These figures are merely schematic representations based on convenience and the ease of demonstrating the existing art and/or the present development, and are, therefore, not intended to indicate relative size and dimensions of the assemblies or components thereof.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

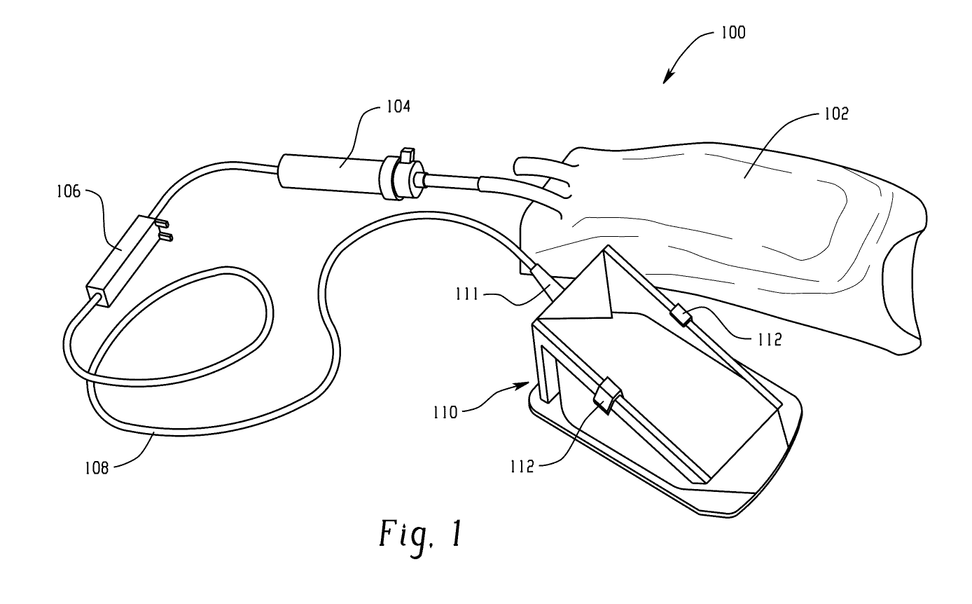

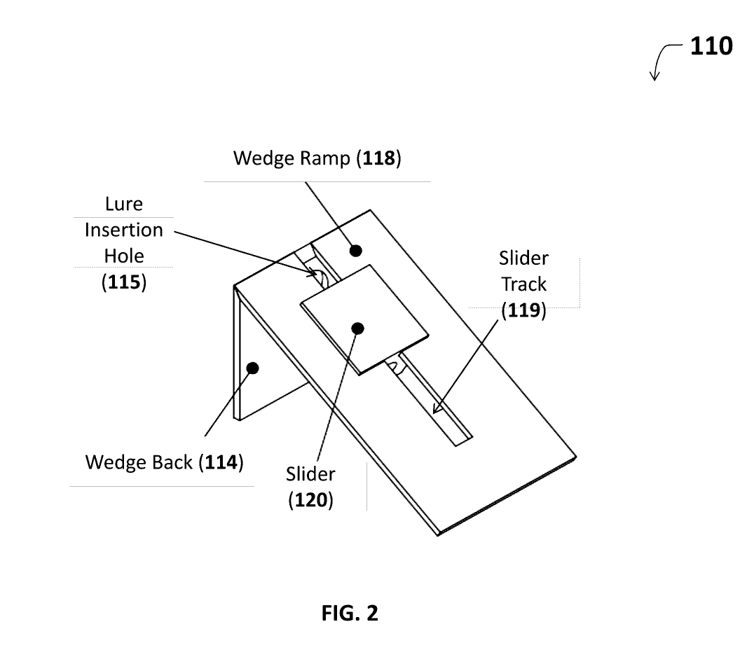

With reference to FIG. 1, a first exemplary system 100 for subcutaneous hydration includes an infusion bag 102 attached to a drip chamber 104. The infusion bag 102 may contain a measured amount, e.g., 500 mL or more, of saline solution or any other isotonic crystalloid sterile hydration fluid known to one having ordinary skill in the art. The infusion bag 102 may be placed above the head of a patient (not pictured) so that gravity aids the outward flow of hydration fluid. The drip chamber 104 allows for a constant drip or flow rate to be established. A tubing 108 extends from the drip chamber 104 and channels the flow of hydration fluid to a deployment device 110. A roller clamp 106 acts to selectively close or open the flow of hydration fluid through the tubing 108 based on the decision of a rehydration fluid administrator. The tubing 108 interfaces with the deployment device 110 at a tubing-lure-needle-connection 111. Optional slider locks 112 affect the operability of the deployment device 110, which will be described in further detail in FIG. 2.

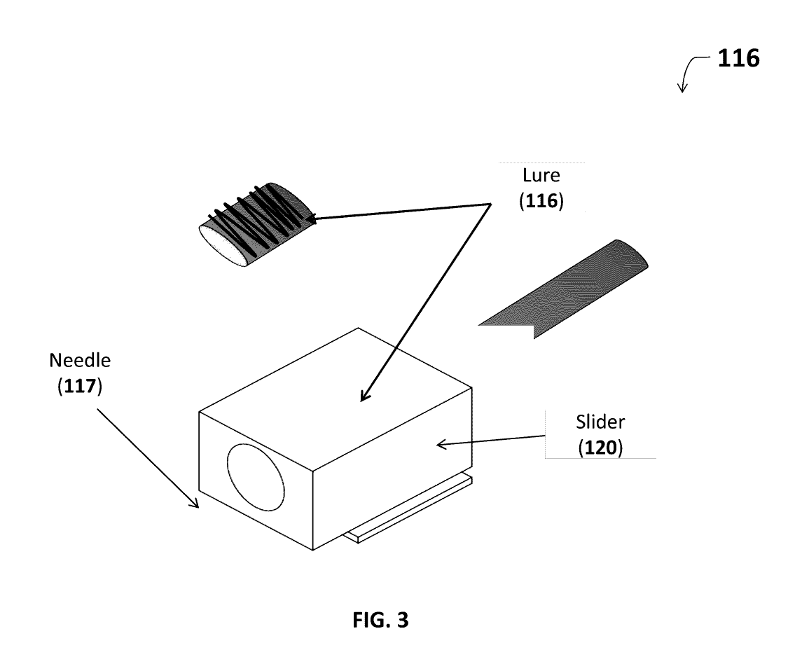

With reference to FIG. 2 and FIG. 3, the deployment device 110 according to a first exemplary embodiment is comprised of a wedge back 114 which is connected to a wedge ramp 118 at a predefined angle. The wedge ramp 118 includes a slider track 119 which enables a slider 120 to translate along the plane of the wedge ramp 118. The wedge back 114 includes a lure insertion hole 115 (view partially blocked) for inserting a lure 116 which may be attached to a needle 117. The lure also travels within the slider 120, where the slider 120 is removably attachable to the wedge ramp via the slider track 119.

The wedge back 114, wedge ramp 118, and slider 120 may be made of acrylic or other low cost material. The lure 116 may be a catheter lure that is approximately 0.75 inches in diameter. The needle 117 may be a 0.5 inch in length needle with a 24 gauge diameter.

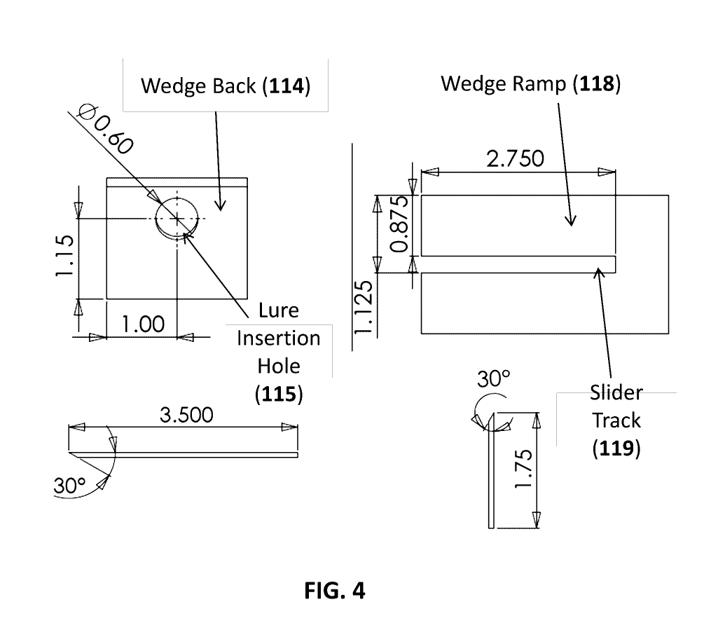

With reference to FIG. 4, the predefined angle between the wedge back 114 and wedge ramp 118 and exemplary length and width dimensions for the wedge back 114, lure insertion hole 114, and wedge ramp 118 according to one embodiment of the deployment device 110 are illustrated in FIG. 4. It should be noted that while 30 degree angles are indicated in FIG. 4, angles between approximately 10 and 30 degrees is also contemplated.

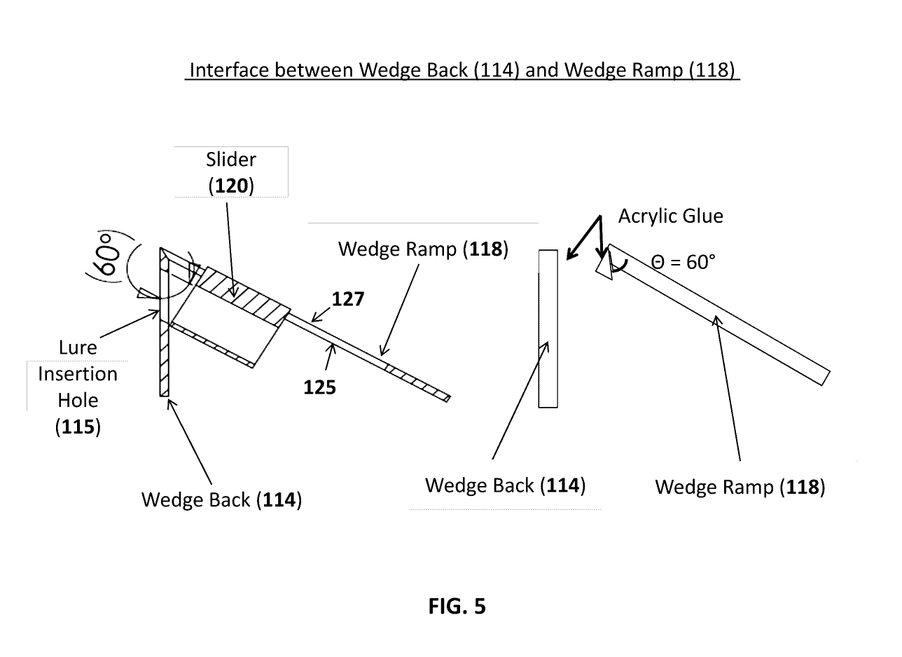

With reference to FIG. 5, the interface between the wedge back 114 and wedge ramp 118 is illustrated. Wedge back 114 and wedge ramp 118 may intersect at approximately a 60 degree angle and may be further be held together by acrylic glue. Other adhesives may be used as known by one having ordinary skill in the art. The majority of slider 120 is located on the underside 125 of wedge ramp 118 once fit within the slider track 119 (not shown).

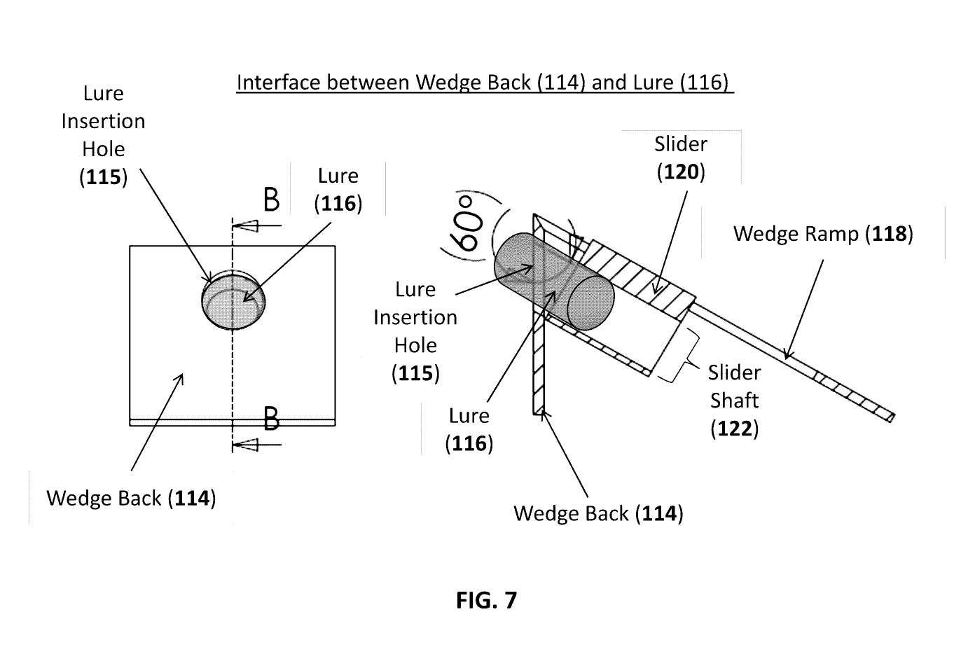

With reference to FIG. 7, the interface between the slider 120 and the lure 116 reveals that the slider 120 may both encompass the lure 116 within shaft 122 and also direct the lure 116 in and out of lure insertion hole 115 in wedge back 114 as slider 120 translates along the tracks 119 cut into the wedge ramp 118.

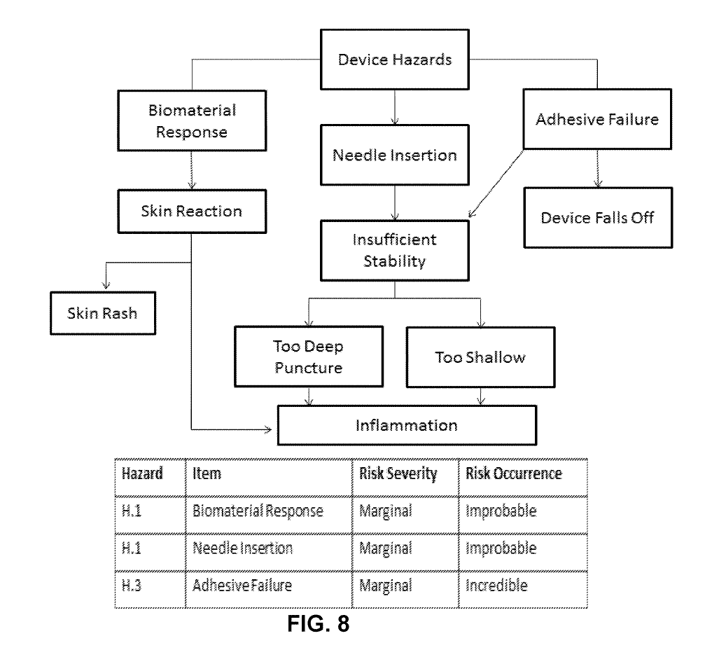

With reference to FIG. 8, there are three classes of common hazards associated with deployment device 110 and similar subcutaneous hydration devices. These risks generally include (1) unfavorable biomaterial responses, such as skin reaction, skin rash, and inflammation, (2) unstable needle insertion, whether too deep, two shallow, or at an improper angle, as well as (3) adhesive failures, which means the device may fall off the skin or fail to properly mount during deployment. Considering these inherent risks for subcutaneous delivery devices, device 110 may incorporate novel biomaterials of novel combinations of biomaterials that minimize reactions and/or rashes. Device 110 may further use a predefined injection angle and or other mechanisms which ensure that puncture is not too deep or shallow even when administered by non-clinically trained technicians. Device 110 may additionally use an appropriate kind and number of adhesive materials to maintain consist mounting of device 110 during injection and administration of hydration fluids.

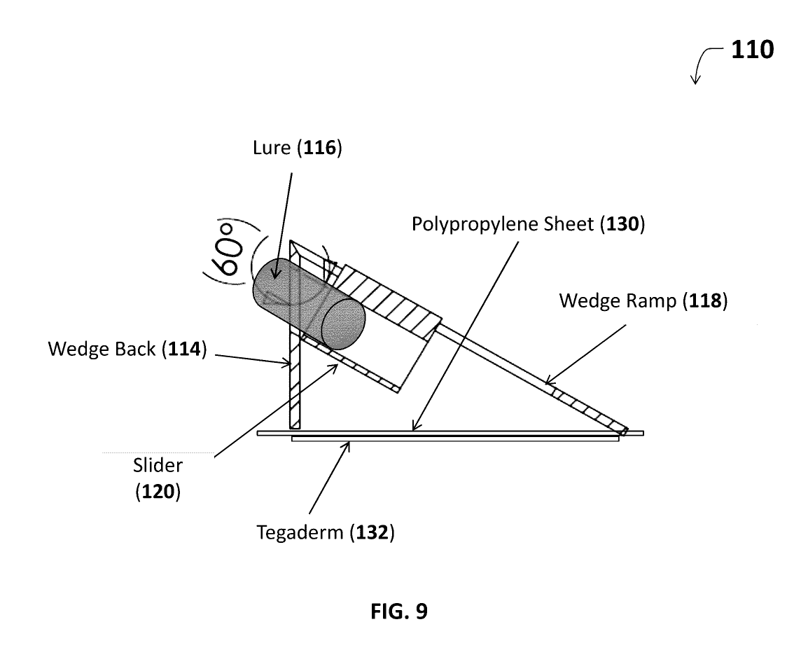

With reference to FIG. 9, the deployment device 110 may be also be envisioned as resting upon a polypropylene sheet 130 with tegaderm adhesive 132 subsequently below for adhering to human skin. These antimicrobial and otherwise biocompatible materials 130, 132 allow for the deployment device 110 to be well adhered to the skin, and therefore promote injection stability, while also not causing an adverse bodily reaction.

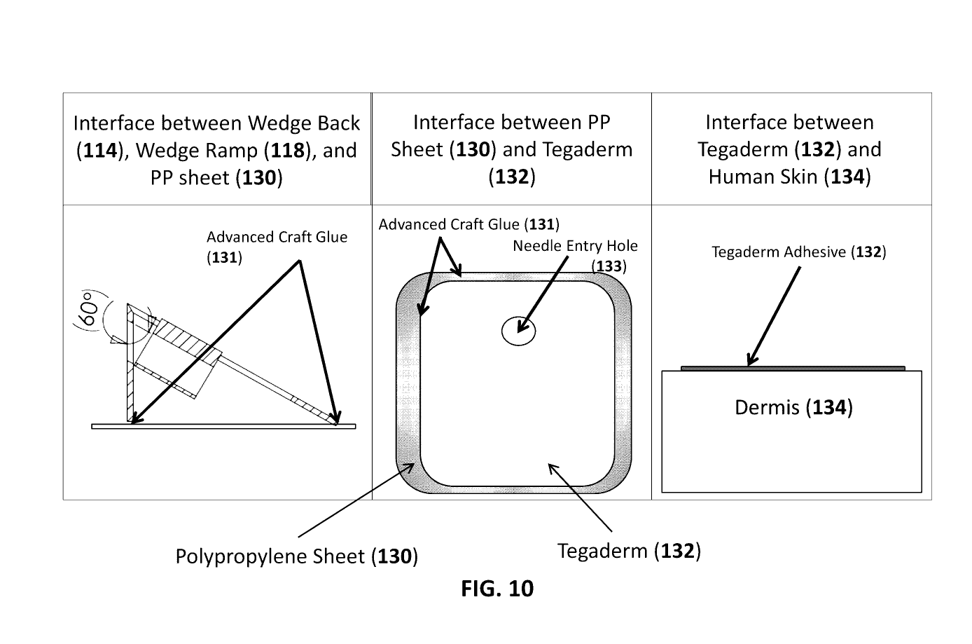

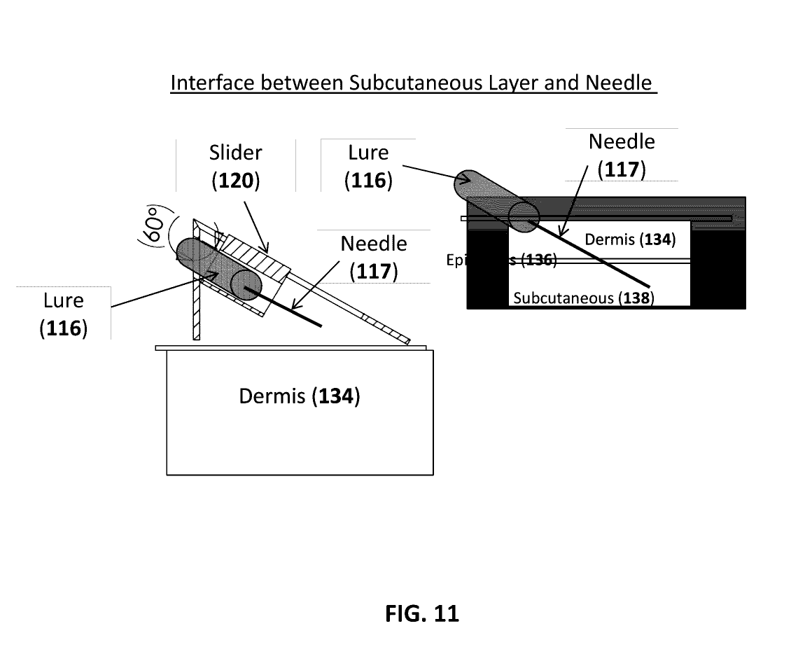

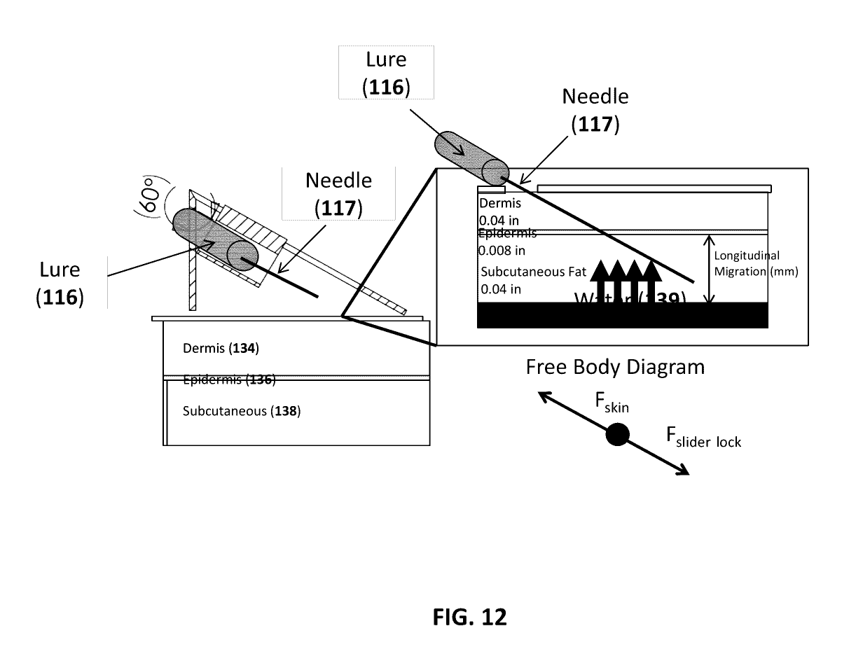

With reference to FIGS. 10-12, the wedge back 114 and wedge ramp 118 may be adhered to the polypropylene sheet 130 referenced in FIG. 9 by advanced craft glue 131, acrylic glue, or other similar adhesives known to one having ordinary skill in the art. In order for a needle 117 attached to the lure 116 to be inserted into a subcutaneous region of human skin (shown in FIGS. 11-12), the needle 117 may have to pass through both the polypropylene sheet 130 and tegaderm adhesive 132. Accordingly, a needle entry hole 133 may be fashioned in the sheet/adhesive 130, 132 at the point of needle insertion into the skin to facilitate injection. Tegaderm adhesive 132 may still be used for stabilizing the deployment device 110 on the dermis 134 without blocking insertion of needle 117 through use of the entry hole 133.

With reference to FIG. 11, the needle 117 is inserted further into the dermis 134 by translating the slider 120 (and encompassed lure 116) towards the dermis 134. The needle 117 will pass through a thin epidermis layer 136 before entering the subcutaneous layer 138 of skin.

With reference to FIG. 12, FIG. 1, and FIG. 2, once the lure 116 and associated needle 117 have penetrated into the subcutaneous layer 138, the lure/needle 116, 117 may be locked into position by locking the slider 120 with at least one slider lock 112. The slider lock 112 places a downward force (F_{slider lock}) on the slider 120, which also experiences a repelling force from the skin (Fₛₖᵢₙ). As water is expelled into the subcutaneous later 138, the skin force Fₛₖᵢₙ becomes larger, and may overcome F_{slider lock}, which tends to vary the position of the needle 117 within the subcutaneous layer 138. The distance of potential needle 117 migrations within the subcutaneous layer 138 has been indicated in FIG. 12 with a longitudinal migration distance.

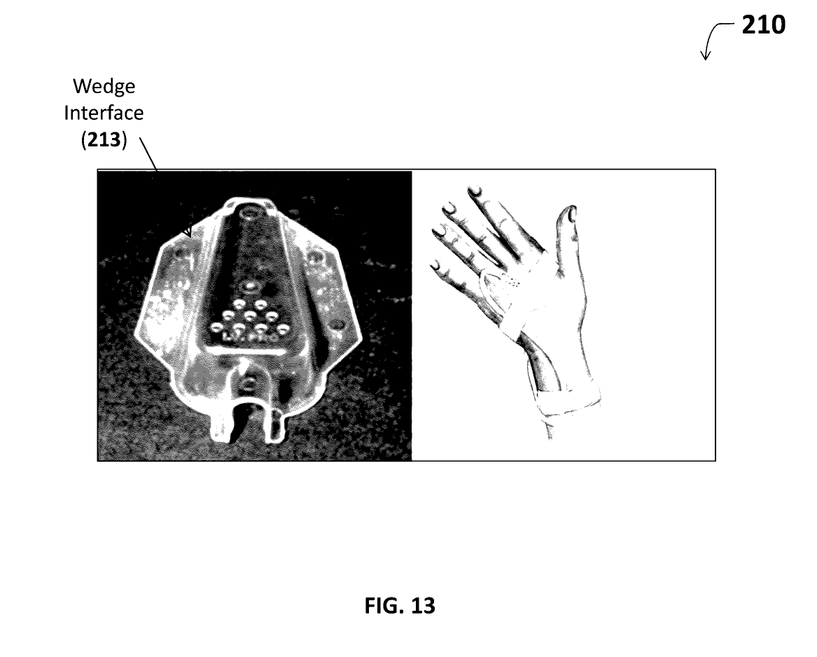

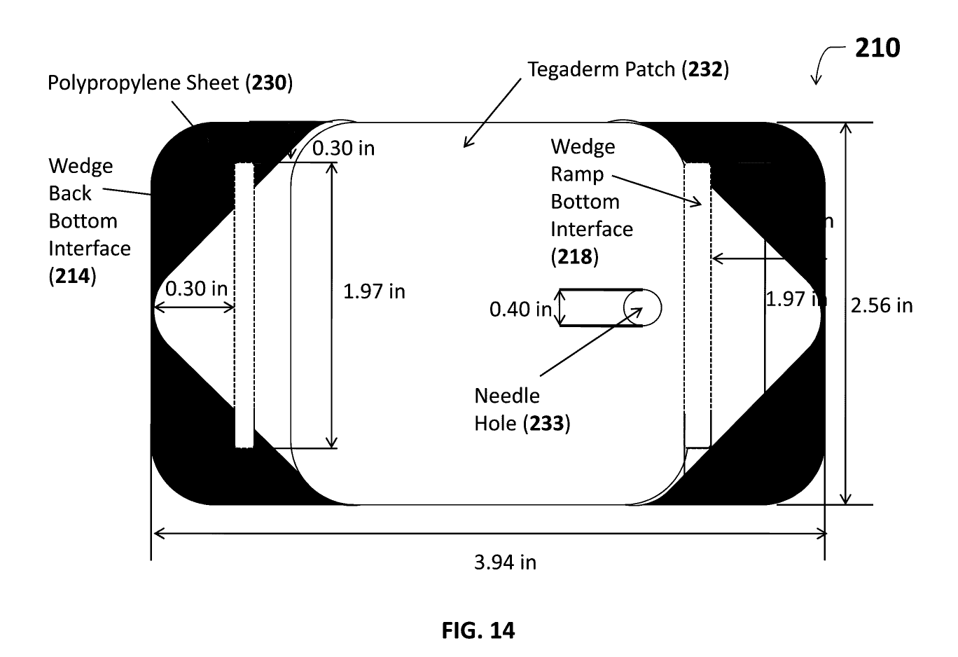

With reference to FIG. 13 and FIG. 14, a deployment device 210 according to a second exemplary embodiment is a smaller device 210 which can fasten to a patient's hand or other small region. The deployment device 210 employs similar injection principles as device 210 however is manufactured differently to provide a smaller integrated wedge interface 213, made up of a wedge back bottom interface 214 and wedge ramp bottom interface 218. The interfaces are placed upon a polypropylene sheet 230 with and tegaderm patch 132, which may be non-rectangular, or take on other shapes as necessary for adhering to different areas of skin. A needle hole 233 provides a point of entry for a needle 217 (not shown) through the polypropylene sheet 230 and tegaderm patch 232 into the skin.

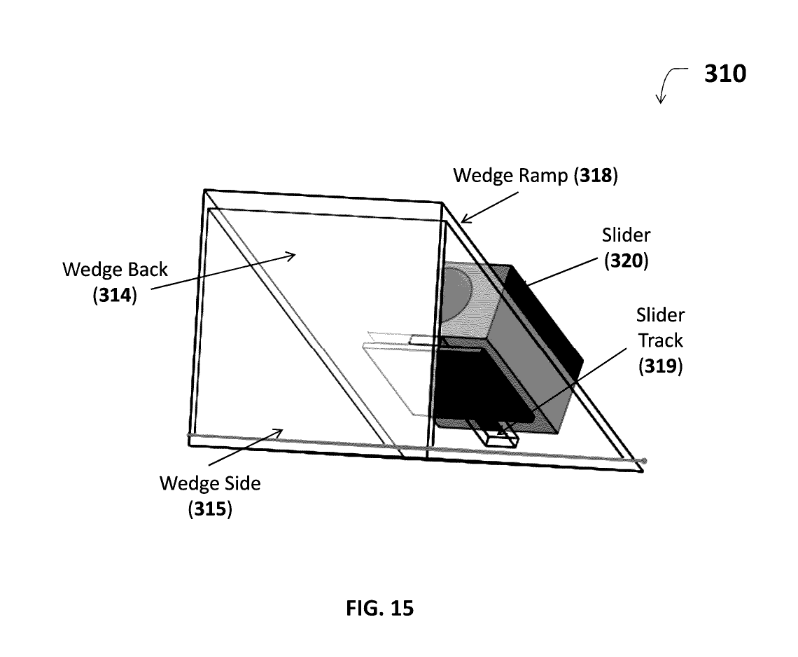

With reference to FIG. 15, a deployment device 310 according to a third exemplary embodiment includes a three wedge pieces: a wedge back 314, wedge side 315, and wedge ramp 318. A slider 320 translates along a slider track 319 cut into the wedge ramp 318, where the track 319 which is relatively smaller than slider track 119 from deployment device 110. All wedge pieces 314, 315, 318, may be constructed from acrylic or other low cost materials as are known to one having ordinary skill in the art.

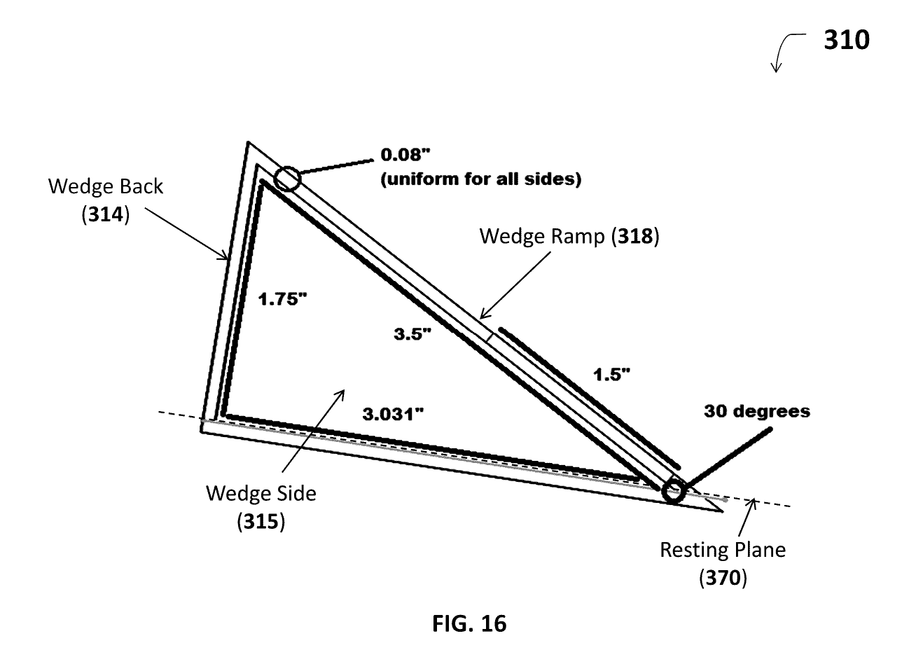

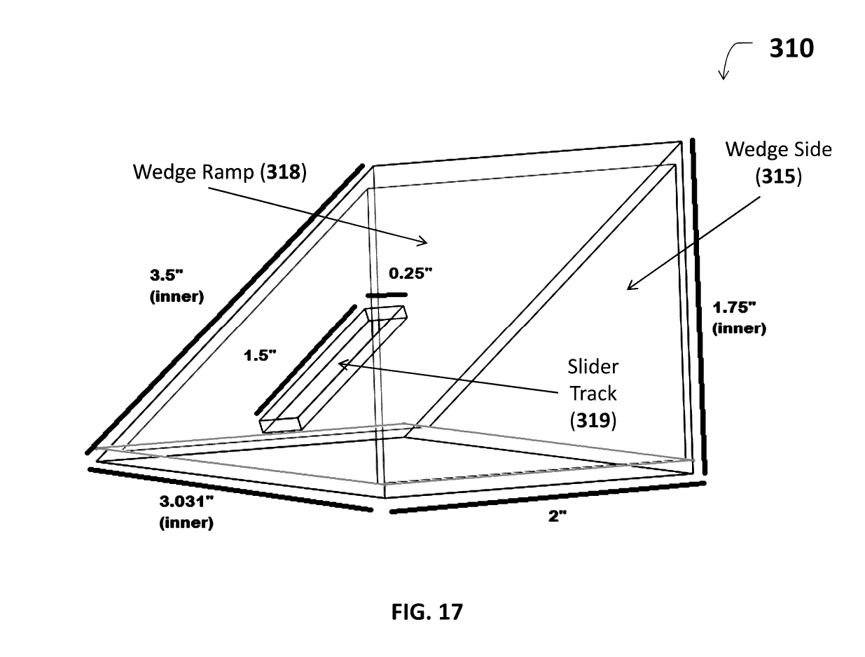

With reference to FIG. 16 and FIG. 17, exemplary dimensions for the wedge pieces 314, 315, 318, slider track 319, and an approximate 30 degree interface angle between wedge ramp 318 and a plane 370 upon which the device 310 rests, are illustrated.

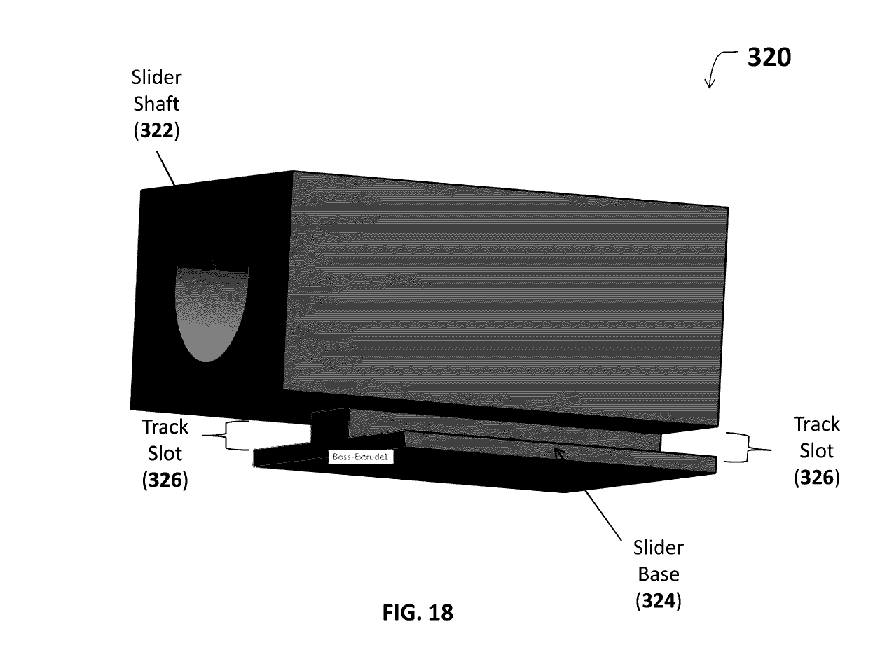

With reference to FIG. 18, a slider 320 fit for deployment device 310 is illustrated. Slider 320 includes a slider base 324 with two track slots 326 for removably attaching slider 320 to slider track 319. Slider slots 326 enable slider 320 to translate along wedge ramp 318, similarly to slider 120 from deployment device 110 described in FIG. 2.

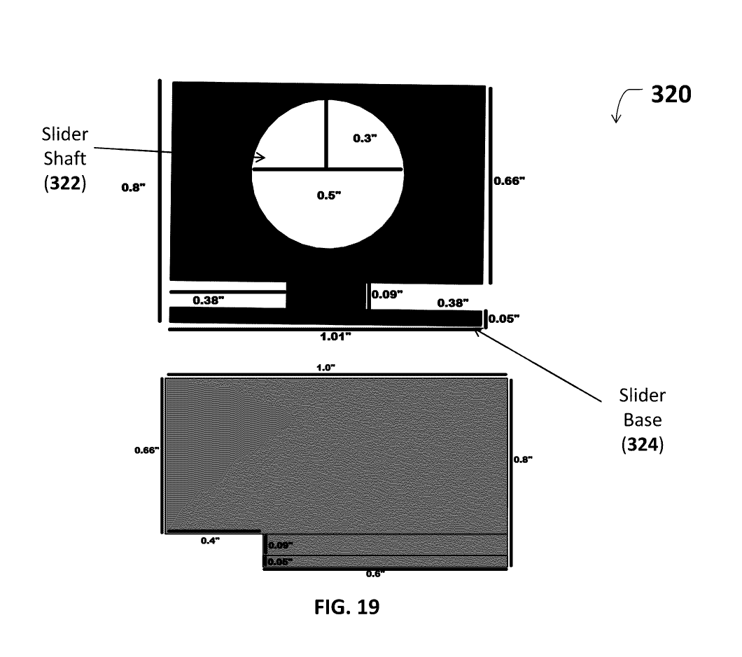

With reference to FIG. 19, exemplary dimensions for slider 320 are illustrated. Slider 320 additionally includes a slider shaft 322 for inserting a lure 316 (not shown) with or without associated needle 317 (not shown). Exemplary dimensions of the slider shaft 322 are indicated.

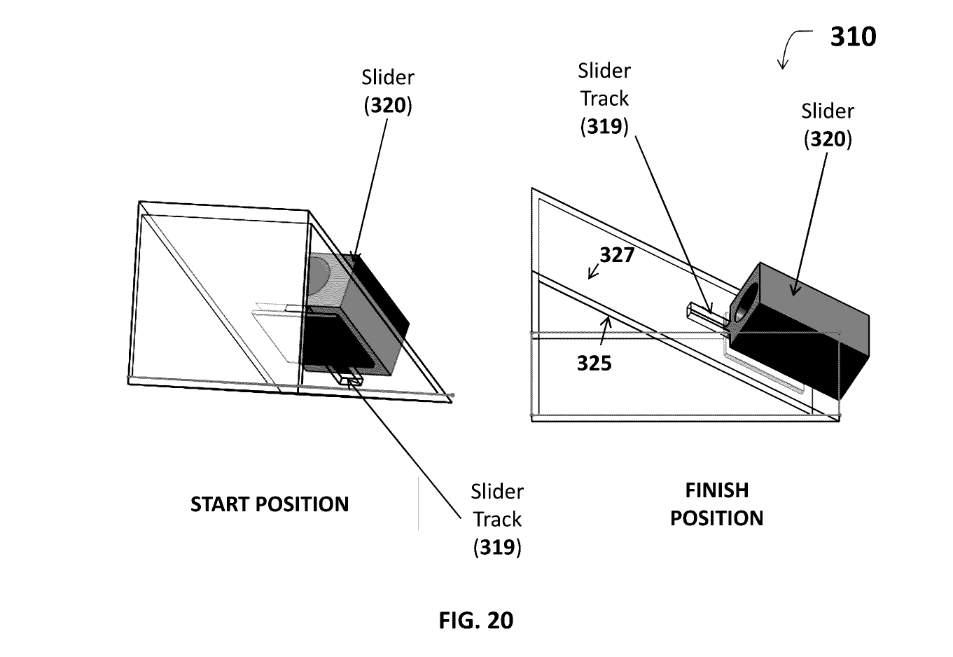

With reference to FIG. 20, the deployment device 310 is shown in both a start and finish position, where if the slider 320 included a lure 316 with associated needle 317, device 310 would be fully deployed into the skin at the “finish position.” To undeploy device 310, slider 320 would be translated along slider track 319 until it rests at the indicated “start position” configuration.

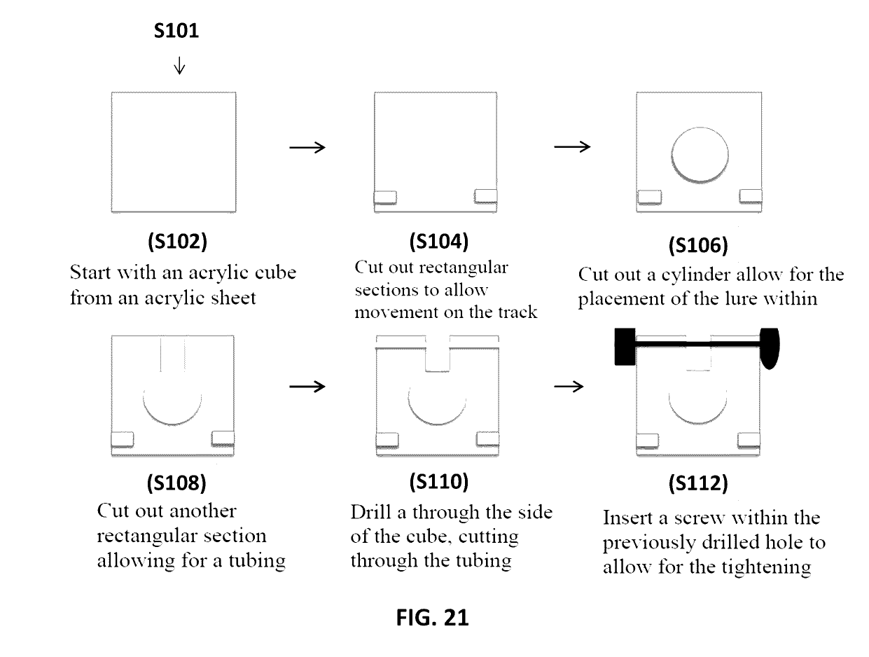

With reference to FIG. 21, a method S100 for forming a low-cost acrylic slider starts at S101. At S102, an acrylic cube is cut from an acrylic sheet. This cube may be 1 inch cubed in dimensions. At S104, rectangular sections are cut from the acrylic cube to allow for movement on a track. At S106, a cylinder is cut from the acrylic cube to allow for placement of a lure within. The cylinder may be of radius 0.25 inches and cut centrally within the cube. At S108, a rectangular section is cut from the acrylic cube to allow for tubing. At S110, a hole is drilled through the side of the cube, cutting through the tubing. At S112, a screw is inserted through the previously made drilled hole to allow for tightening.

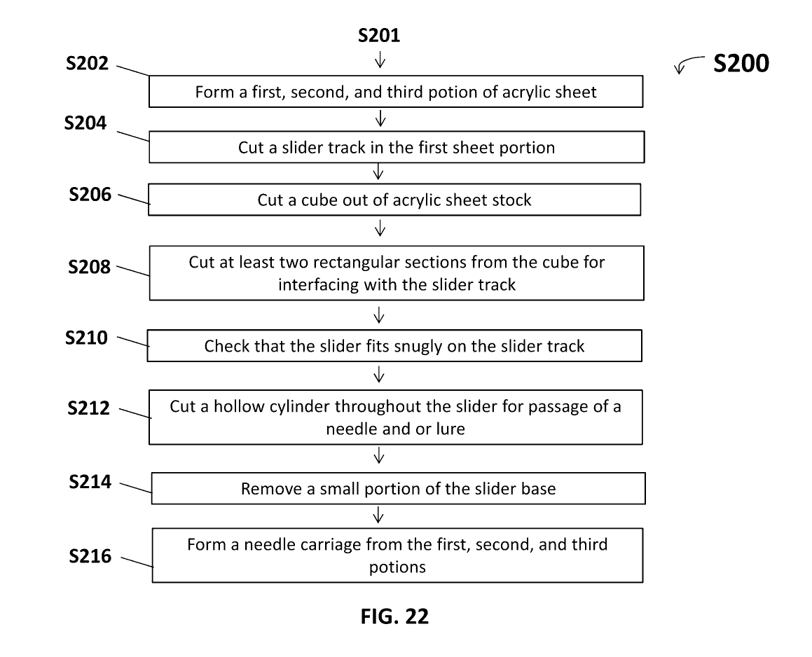

With reference to FIG. 22, a method S200 for forming a low-cost acrylic deployment device starts at S201. At S202, first, second, and third portions of acrylic sheet are cut from acrylic sheet stock. These sheets may represent the horizontal, vertical, and hypotenuse distances within a wedge with dimensions 3.031×2 inch+/−0.1 inch, 2 inch×1.75 inch+/−0.1 inch, and 3.5 inch×2 inch+/−0.1 inch, respectively. At S204, a slider track is cut in the first sheet portion. At S206, a cube is cut out of acrylic sheet stock. At S208, at least two rectangular sections are cut from the cube for interfacing with the slider track. At S210, a check is performed to see that the slider fits snugly on the track. At S212, a hollow cylinder is cut from the slider for passage of a lure and or needle. At S214, a small portion of the slider base is removed. Removal of this portion allows for the slider to reach its desired final position. Further modifications to the slider as disclosed in method S100 may be further incorporated into method S200. At S216, a needle carriage is formed from the first, second, and third acrylic sheet portions. This step may include maintaining an angle of approximately 30 degrees, +/−2 degrees, between acrylic sheet portions which form the smallest angle in the wedge needle carriage.

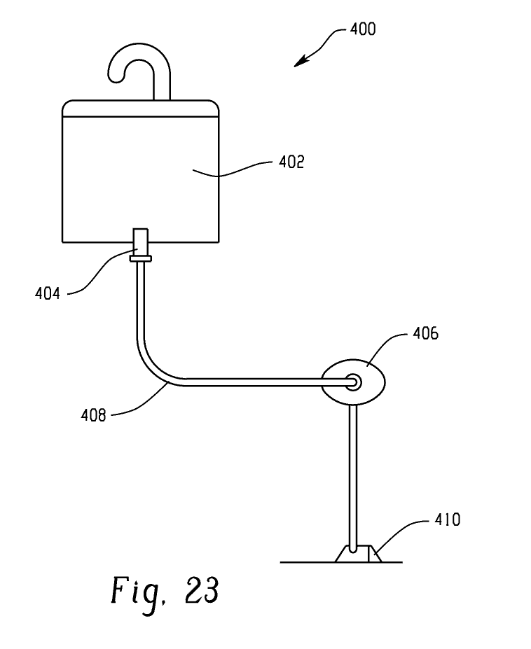

With reference to FIG. 23, a system 400 for providing subcutaneous hydration according to a second exemplary embodiment. System 400 may be a fully integrated system which may provide for facile introduction of fluids into a subcutaneous space of human skin where they absorb into the microvasculature, providing volume replacement, without the need for a skilled operator to get intravenous access. System 400 includes an infusion bag 402 which provides hydration fluid to a deployment device 410 via tubing 408. Infusion bag 402 may include an integrated hanger, as illustrated, for hanging the bag 402 above the head of a patient to make use of gravitational forces in expelling fluids. Instructions may be located on infusion bag 402 to make operation of the system 400 easier to non-clinically trained administrators. The infusion bag 402 would include a measured amount, e.g. 500 mL, of isotonic crystalloid sterile fluids as known to one having ordinary skill in the art. An iodine or similar prep kit (not shown) used for cleaning the skin before injection may be packaged with the infusion bag 402, with instructions for use of the iodine prep kit printed on the infusion bag 402.

The tubing 408 may be pre-connected to the infusion bag 402 to avoid potential confusion for non-skilled operators. The tubing 408 runs through a squeezable bulb 406 before passing to the deployment device 410. The squeezable bulb 406 acts as a priming chamber for flushing and priming the tube 408. The bulb 406 may contain a one-way valve. The tubing 408 may be pre-connected to a needle 417 integrated into the deployment device 410. The needle 417 may be a medium bore needle, e.g. 24 gauge, for general applications however can take on different dimensions as known to one having ordinary skill in the art when used for specialized applications.

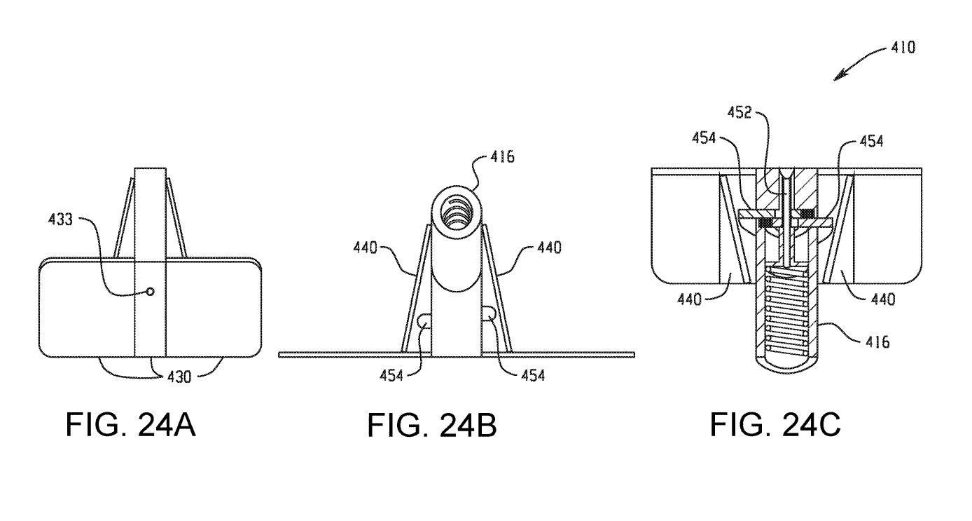

With reference to FIG. 24A, FIG. 24B, and FIG. 24C, the deployment device 410 in an undeployed mode is illustrated in a bottom cutaway view in FIG. 24A, a rear cutaway view in FIG. 24B, and a top cutaway view in FIG. 24C. Deployment device 410 includes a base 430 for resting on the skin, which may be flexible or hinged depending on the material used to construct the base 430. The base 430 may include an antimicrobial material and/or water proof adhesive (not shown) for interfacing with the skin of a patient. A needle aperture 433 is located in the base 430 so that a needle 417 (not shown) located in a spring-loaded barrel 416 may pass through a needle slot 452 in barrel 416 and aperture in base 430 to puncture the skin once device 410 is placed in a deployed mode. The spring-loaded barrel 416 is also angled at an appropriate angle for safe injection, e.g. approximately 30 degrees. The barrel also houses the tubing 408 (not shown) which may be pre-connected to the needle 417. Wings 440 located on either side of barrel 416 are used for pushing the two sides of the base downward in order to pull up a tent of skin from a patient used for injection, and are further used to trigger actuators 454 which place device 410 in a deployed mode for injection activity.

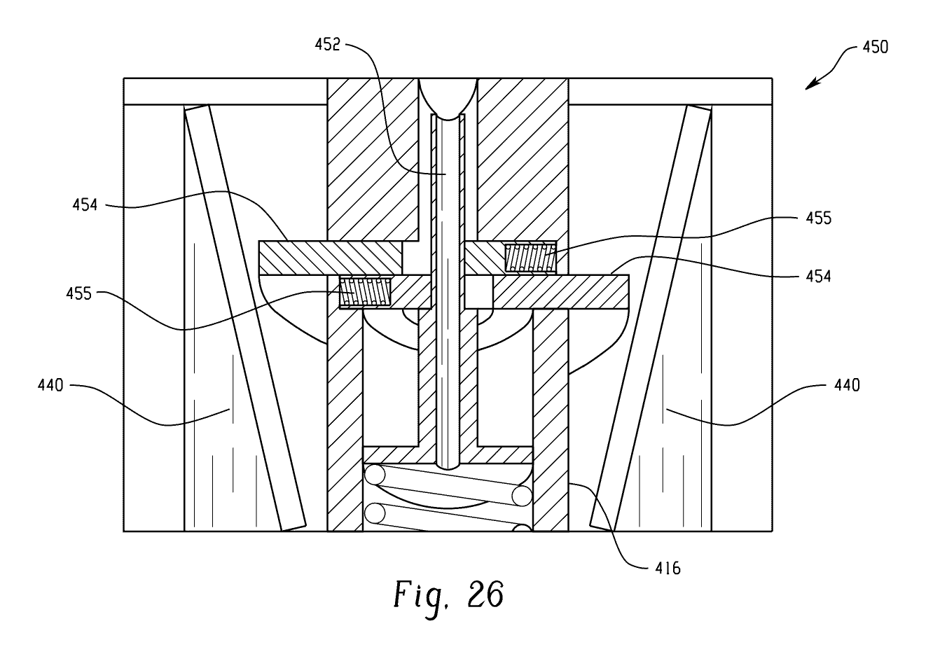

One novel feature of deployment device 410 is the increased injection safety provided by barrel 430, wings 440, and actuators 454. The needle 417 will only deploy as a tent of skin is “pinched” up between the wings 440. This creates a space into which the needle is automatically and safely deployed once the wings 440 are squeezed enough to activate actuators 454 which unlock a firing mechanism 450 (described in FIG. 26 below) within the barrel to deploy the needle 417.

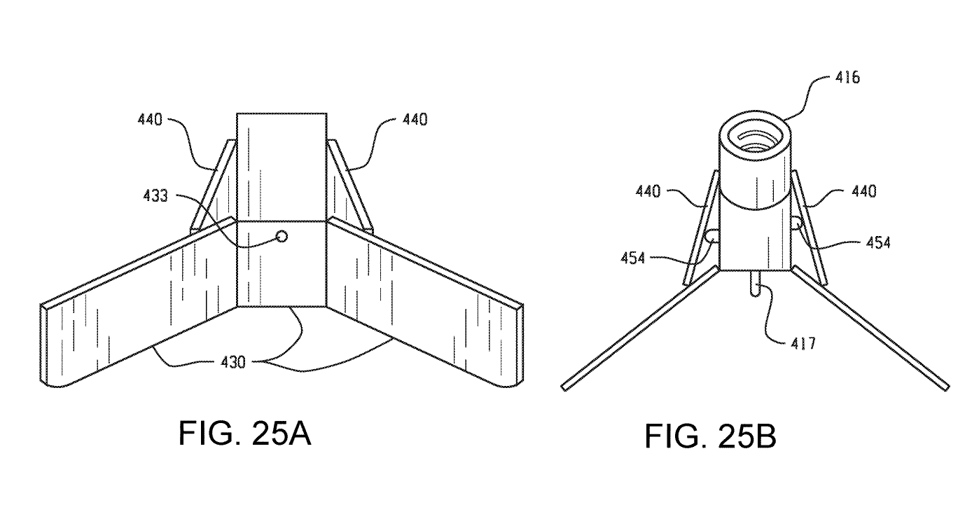

With reference to FIG. 25A and FIG. 25B, the deployment device 410 in a fully deployed mode is illustrated in a bottom cutaway view in FIG. 25A and rear cutaway view in FIG. 25B. Wings 440 have been adequately squeezed in order to engage the actuators 454. The needle 417 has been automatically deployed out of the aperture 433 located in the base 430 and can be seen coming out of the barrel 416 in the (b) rear cutaway view.

With reference to FIG. 26, the firing mechanism 450 when the deployment device 410 is in the undeployed mode has been illustrated in greater detail. Actuators 454 may be connected to springs 455 which reside within barrel 416. When actuators 454 are sufficiently depressed by squeezing wings 440, the wings touch the actuators 454 and depress the actuators 454 and attached springs 455. As the actuators 454 are depressed, the needle slot 452 becomes unblocked so that a needle 417 (not shown) may be automatically deployed through the needle slot 452 to partially exit the barrel 416 and inject into the skin. Needle 417 is still partially held within the barrel 416, at least at a needle hub (not shown), to maintain the general angle of injection and to allow slight vertical pivot to accommodate skin movement.

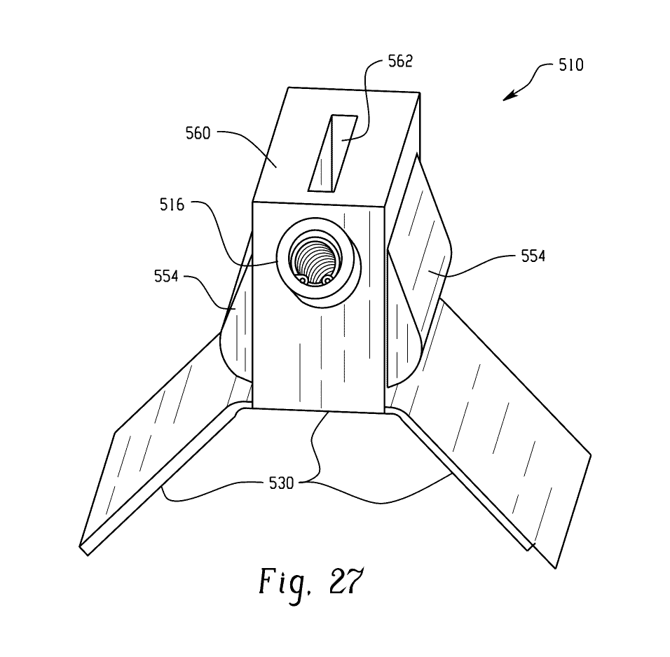

With reference to FIG. 27, a 3D model of a deployment device 510 according to a fourth exemplary embodiment is shown. Deployment device 510 is modeled closely after device 410, however includes many notable improvements. Such improvements may include a more ergonomic shape for actuators 540 that resemble buttons and may promote squeezing. A casing 560 may be fit over a barrel 516 so that the barrel does not extrude far from the device. The casing 560 may include a slider track 562 used in conjunction with a slider 520 (not shown) to retract a needle 517 after it has been deployed. It should be noted that wings 540 (not shown) may emanate from near the top of the casing 560 and attach to the base 530. The wings 540 would function similar to the wings 440 in device 410, and could be used to depress actuators 554.

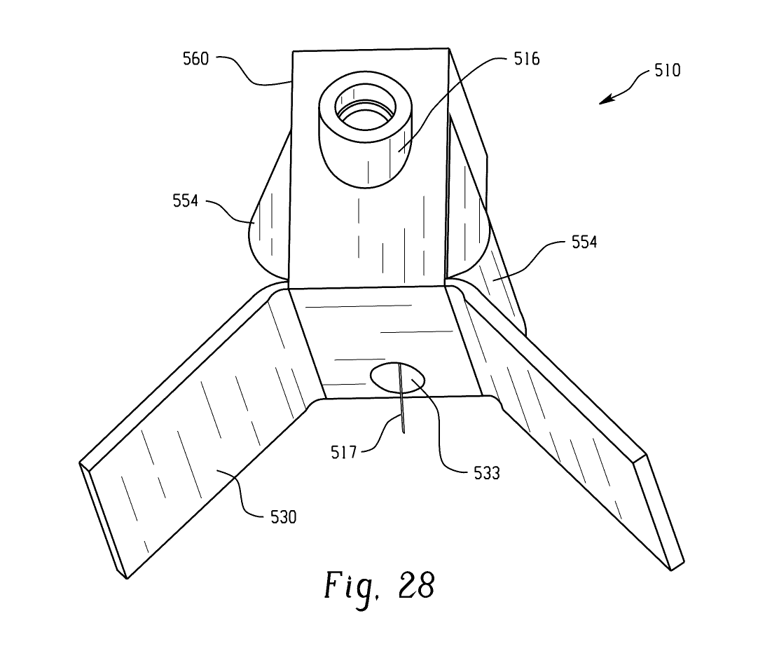

With reference to FIG. 28, the deployment device 510 is shown in a deployed mode perspective view with the needle 517 protruding out of the barrel 516 through a needle aperture 533 located on base 530.

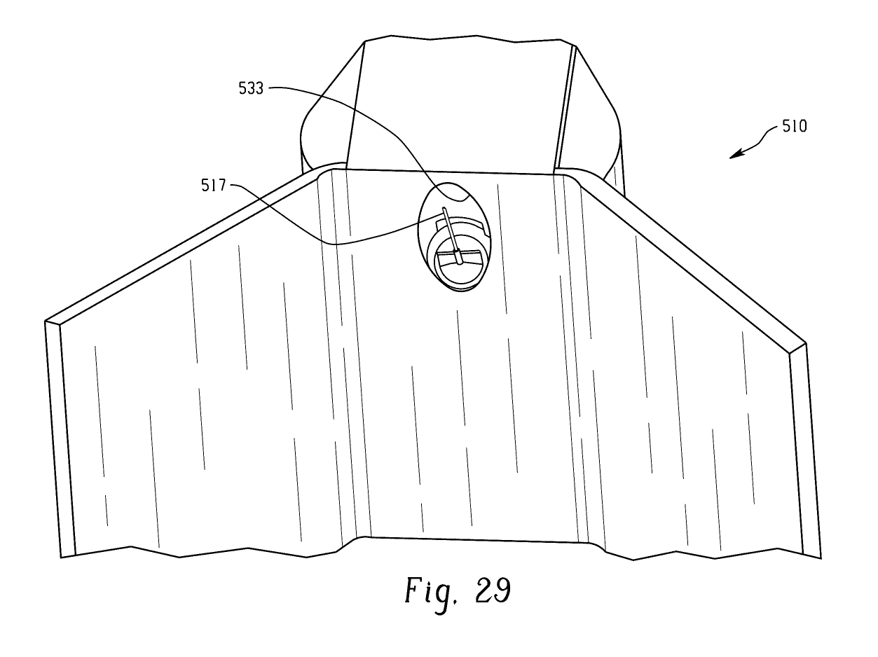

With reference to FIG. 29, the needle aperture 533 located on base 530 may be oblong or take on other beneficial shapes as appreciated by one having ordinary skill in the art to allow the needle 517 to pivot about the hub while deployed into the skin.

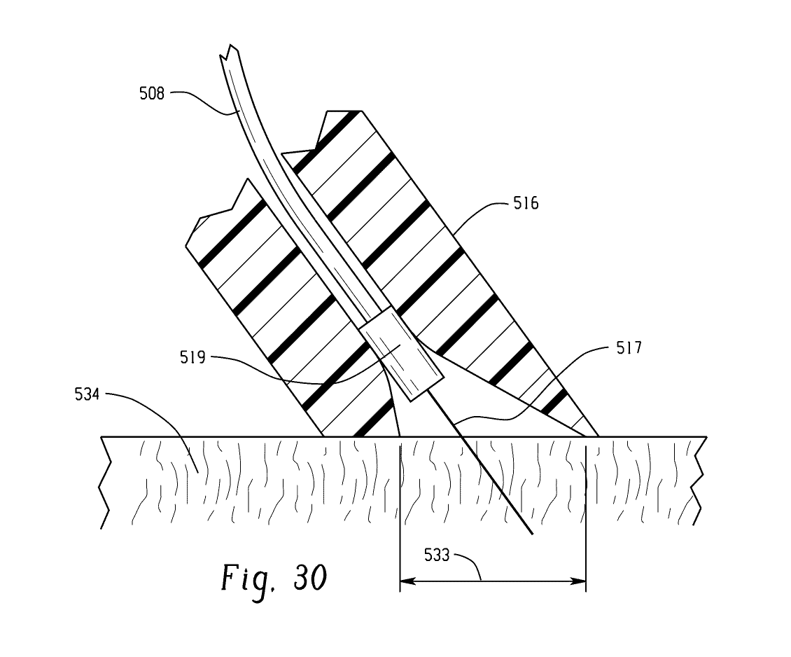

With reference to FIG. 30, needle 517 may be pre-connected to a tubing 508, both of which are partially housed within the barrel 516. The needle 517 once injected into dermis 534 may vary in position, however is held firm within the barrel 516 at least at the needle hub 519. Needle 517 position variance within aperture 533 may be due to expelled hydration fluid within a subcutaneous skin region pushing up against the needle. This concept was previously illustrated by increased force, Fₛₖᵢₙ, in FIG. 12. An oblong shape allows for needle 517 position variance without displacing needle 517 from the barrel 516 or causing unnecessary patient discomfort or tissue trauma.

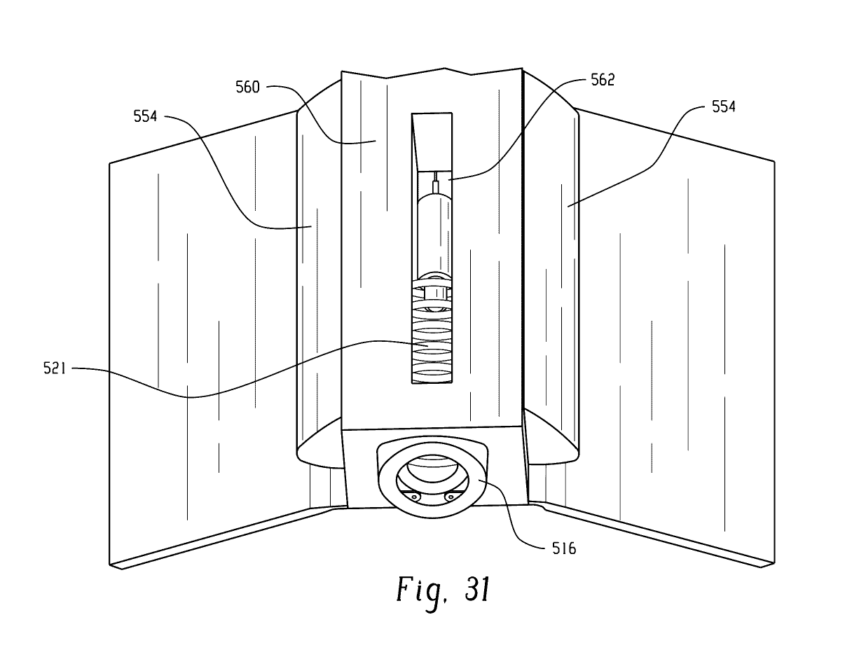

With reference to FIG. 31, the slider track 562 is located above the spring-loaded barrel 516. An appropriate slider 520 (not shown) which fits along slider track 562 may include a snag or similar feature which engages or “catches” the spring 521 within the spring-loaded barrel 516 and retracts the needle 517 attached to the spring 521 when the slider 520 is translated along the slider track 562.

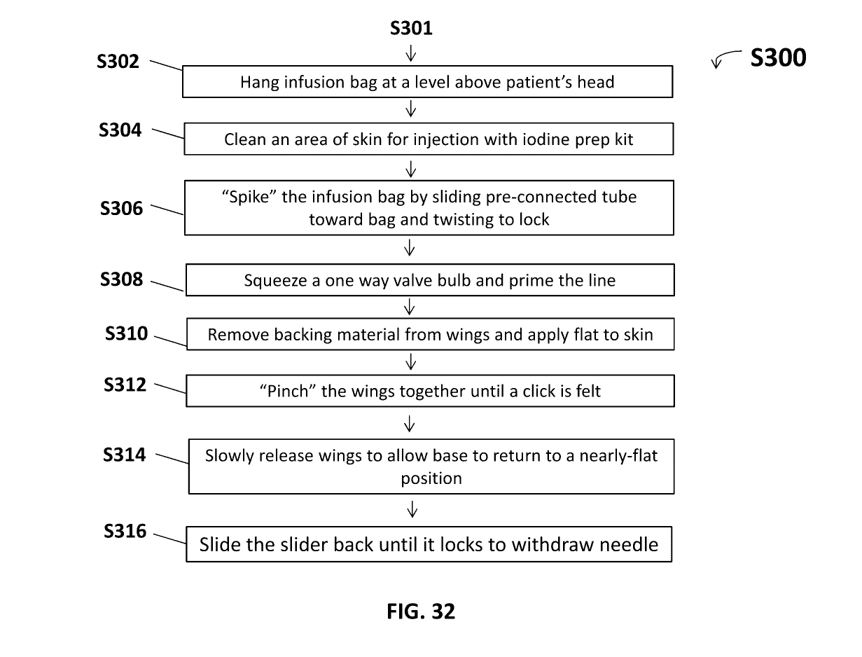

With reference to FIG. 32, an exemplary method S300 for performing subcutaneous rehydration begins at S301. Method S300 may be performed with deployment device 410, 510 or other similar devices. At S302, an infusion bag is hung at a level above a patient's head. At S304, an area of skin is cleaned for injection with an iodine prep kit or the like. In one embodiment, the area of skin cleaned for injection is at least one of a back, thigh, or abdomen bodily area. At S306, the infusion bag is “spiked” by sliding a pre-connected tube towards the bag and twisting to lock. At S308, a one way valve bulb is squeezed to prime the tube. It should be noted that S306 or S308 may be performed in the alternative or in combination to prime the tube. At S310, backing material is removed from wings and applied flat to the skin. At S312, pinch the wings together until a click is felt. This action may pull up a tent of skin and automatically deploy a needle. At S314, wings are slowly released to allow base to return to a nearly-flat position. At S314, a slider is translated until locked in order to withdraw the needle from the patient.

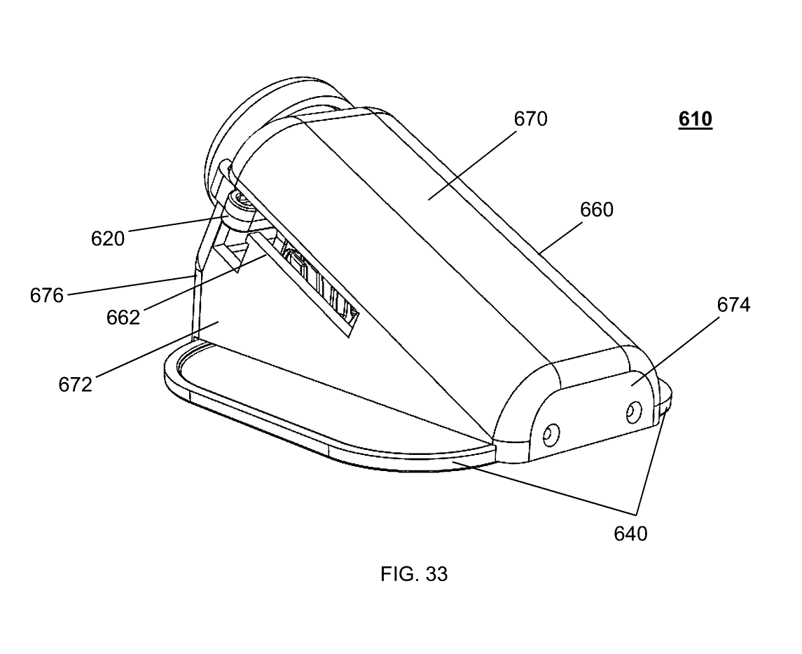

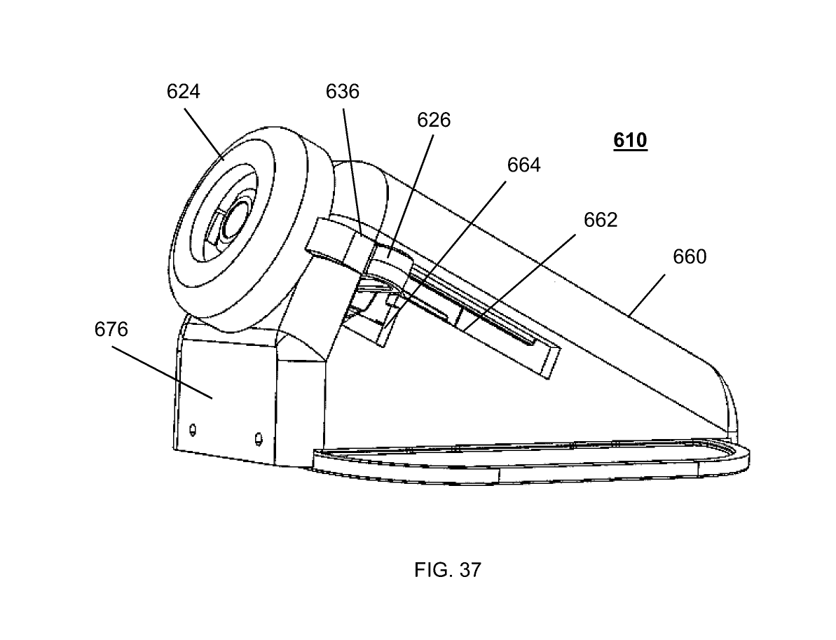

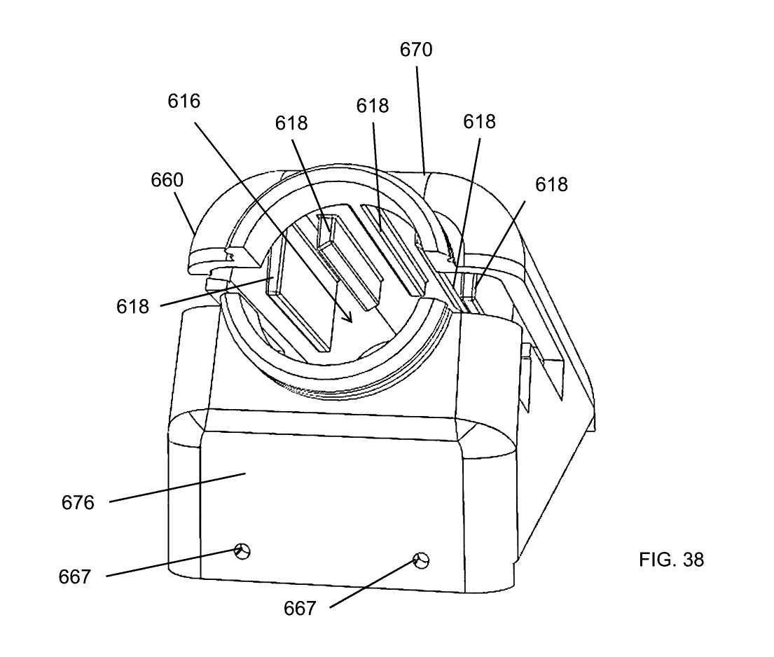

FIGS. 33-41 illustrate a deployment device 610 according to a fifth exemplary embodiment. Deployment device 610 is very similar to device 510. Referring first to FIG. 33, the deployment device 610 is shown in an undeployed mode with wings 640 located on either side of casing 660. The casing 660 includes a slider track 662 used in conjunction with a slider 620 to deploy and retract a needle (not visible). The casing 660 is located above the wings 640, and as will be seen later, the wings are pushed downwards to deploy the needle. The casing includes a top wall 670, two side walls 672, a front wall 674, and a rear wall 676 (see FIG. 37 and FIG. 38).

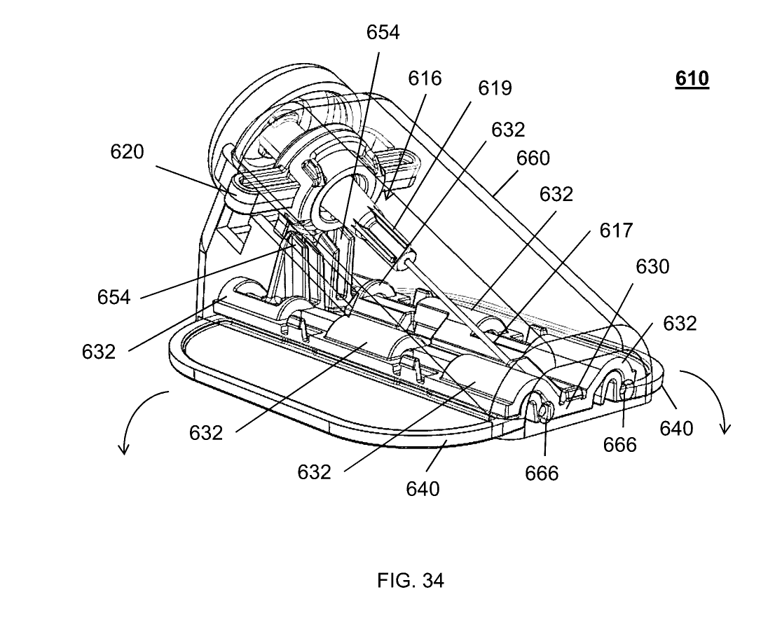

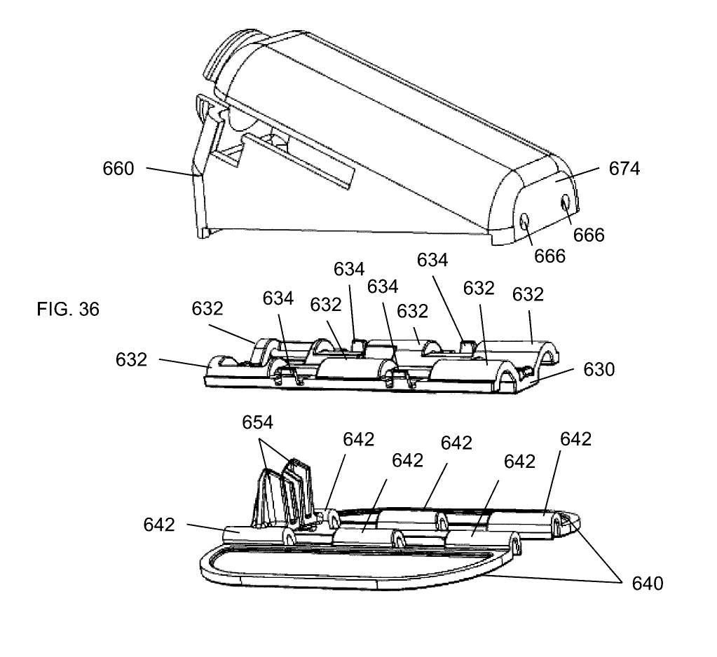

In FIG. 34, the casing 660 is transparent, so the internal components of the device can be more easily seen. Deployment device 610 includes a base 630 for resting on the skin. The casing 660 and the base 630 engage each other, and form a hollow “barrel” 616 within the device. The wings 640 are also shown. The base 630 includes knuckles 632, and the wings include knuckles 642 (these are better seen in FIG. 36). Pins engage these knuckles through apertures 666 in the front wall of the casing 660, so that the wings 640 are hinged relative to the casing 660 and the base 630. The exterior surface of the base 630 may include an antimicrobial material and/or waterproof adhesive (not shown) for interfacing with the skin of a patient. The base can be made from a soft material (e.g. silicone) to facilitate grip.

Also visible in FIG. 34 are a slider 620, a needle hub 619, and the needle 617, which are parts of the firing mechanism. Actuators 654 are also shown, which are seen here in a “vertical” position and preventing the slider 620 from moving downwards (i.e. towards the base). When the wings 640 are pushed downward, they can pull up a tent of skin from a patient, and the needle will be deployed into this tent of skin. Moreover, as the wings 640 are pushed downward, the actuators 654 protruding upward from the wings 640 rotate outward (i.e., to the sides), thereby permitting the needle 617 to be deployed.

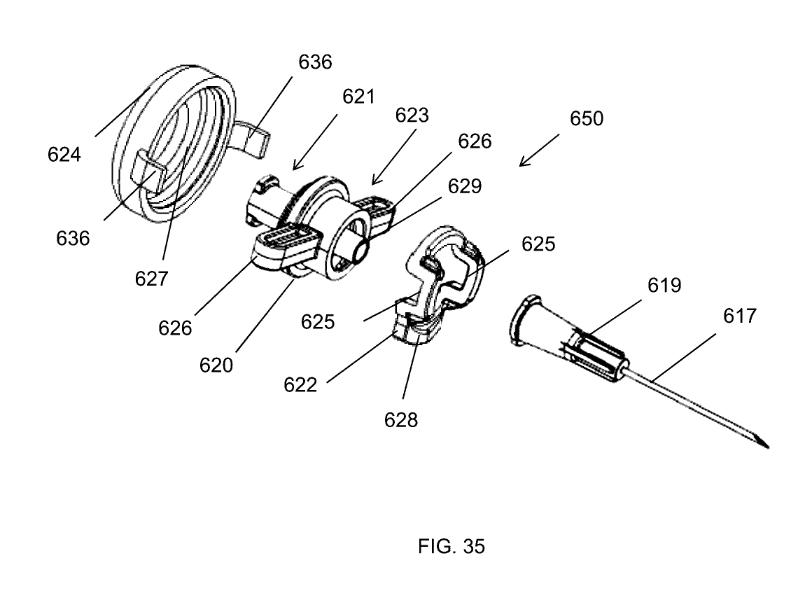

With reference to FIG. 35, the firing mechanism 650 is illustrated in an exploded view. As shown, the firing mechanism 650 generally includes the needle 617, a needle hub 619 for firmly holding the needle within the barrel, the retaining ring 622, a slider 620 to both deploy and retract the needle after it has been deployed, and an end cap 624 which fits on the casing 660 to enclose the barrel 616. The slider 620 includes slider handles 626 which protrude outward therefrom to provide a gripping surface which can be utilized to retract the needle. Although not illustrated in FIG. 35, a spring is also included as part of the firing mechanism 650. The spring is generally positioned between the end cap 624 on one end and the slider 620 on another end.

With reference to FIG. 35, the firing mechanism 650 is illustrated in an exploded view. As shown, the firing mechanism 650 generally includes the needle 617, a needle hub 619 for firmly holding the needle, a retaining ring 622, and a slider 620 to both deploy the needle and to retract the needle back into the casing after deployment. Also shown is an end cap 624 which fits on the casing 660 to enclose the barrel 616. The end cap has projecting walls 636 that will define a rear end of the slider track, as seen later.

The slider 620 is in the shape of a ring, and includes a rear portion 621 and a front portion 623. Slider handles 626 protrude sideways from the slider 620, and provide a grippable surface which can be utilized to retract the needle. A hollow passage 629 passes through the slider from the rear portion 621 through the front portion 623. In use, tubing can be attached to the rear portion 621, so that fluid (e.g. saline or other IV fluid) can be run through the slider 620 and into the needle 617.

The retaining ring 622 is generally received on the front portion 623 of the slider 620 and includes recessed portions 625 which are adapted to fit over the slider handles 626. A stop wall 628 extends from the retaining ring 622 in a downward direction, which will engage the actuators 654 of the wings 640. The retaining ring engages the needle hub 619, securing the needle 617 to the slider 620. It is noted that the retaining ring is not required in all embodiments. The slider 620 can be shaped to both include the stop wall 628 and to provide a structure for securing the needle hub 619.

Although not illustrated in FIG. 35, a spring is also included as part of the firing mechanism 650. The spring is generally positioned between the end cap 624 on one end and the slider 620 on another end. In the retracted position, the spring is compressed and stores energy, and in the deployed position the spring is extended. The spring is biased to be extended.

Referring back to FIG. 34, when the wings 640 are level with or above the plane of the base 630, the stop wall 628 engages the actuators 654 to retain the device 610 in the undeployed position, i.e. the needle 617 is within the casing 660. For example, the actuators 654 act against the bias of the spring (not shown) acting between the slider 620 and the end cap 624.

Although not illustrated in FIG. 35, a spring is also included as part of the firing mechanism 650. The spring is generally positioned between the end cap 624 on one end and the slider 620 on another end.

Referring back to FIG. 34 and to FIG. 35, the rear portion 621 of the slider may partially fit within a recessed portion 627 of the end cap 624. The slider handles 626 extend outside of the casing 660, through the slider tracks 662, for manipulation by a user of the device 610.

Referring now to FIG. 36, the casing 660, the base 630, and wings 640 are illustrated in an exploded view. One or more clips 634 located on the base 630 snap into one or more recesses (not shown) located on an inner surface of the casing 660 such that the casing and base are securely connected to each other. The base 630 also includes one or more knuckles 632 located on either side of the base which align with corresponding knuckles 642 located on the wings 640. Pins, rods, or wires (not shown) run through the knuckles to form a hinge. The front wall 674 of the casing 660 can include apertures 666 for the pins/rods/wires as well. Corresponding apertures 667 are present in the rear wall 676 of the casing 660, as seen in FIG. 38. The actuators 654 extend upwards on the wing 640.

FIG. 37 is an exterior view of the device 610. The rear wall 676 of the casing 660 is visible, along with the end cap 624 attached thereto. The end cap is generally press-fit onto the rear casing. The projecting walls 636 align with the slider track 662. A recessed notch 664 is visible below the slider track 662, and the slider handle 626 extends outwards through the casing into the slider track 662.

FIG. 38 is a rear view of the casing 660, with the end cap removed so the interior of the casing can be seen. Initially, apertures 667 are present on the rear wall 676. Pins/rod/wires pass through this aperture to form a hinge between the wings and the base. The barrel 616 is indicated. Column walls 618 are present within the interior of the casing, shown here extending downwards from the top wall 670 of the casing 660 The column walls 618 together with the slider track 662 define a pathway for the firing mechanism to travel. Although not shown in this embodiment, column walls may also extend upwards from the base if desired to help define the pathway.

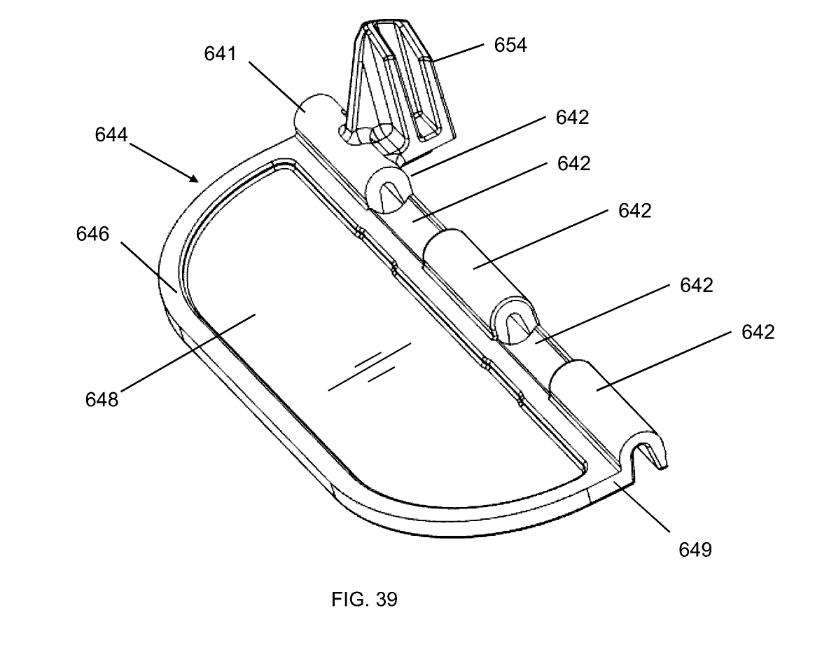

FIG. 39 is a perspective view of a wing 640. The wing has a rear end 641 and a front end 649. The wing includes a frame 644. One part of the frame includes knuckles 642 which make up part of a hinge with the base. A spar 646 extends away from the knuckles, and is pushed downwards by the user. As shown here, the spar 646 and knuckles 642 surround a surface 648, though this surface is optional. The spar 646 defines a plane, and the actuator 654 extends upwards from this plane. As seen here, the actuator 654 is on one side of the knuckles 642, and the spar is on the other side of the knuckles 642. The actuator 654 is shown here on the rearmost of the knuckles, though this is not significant.

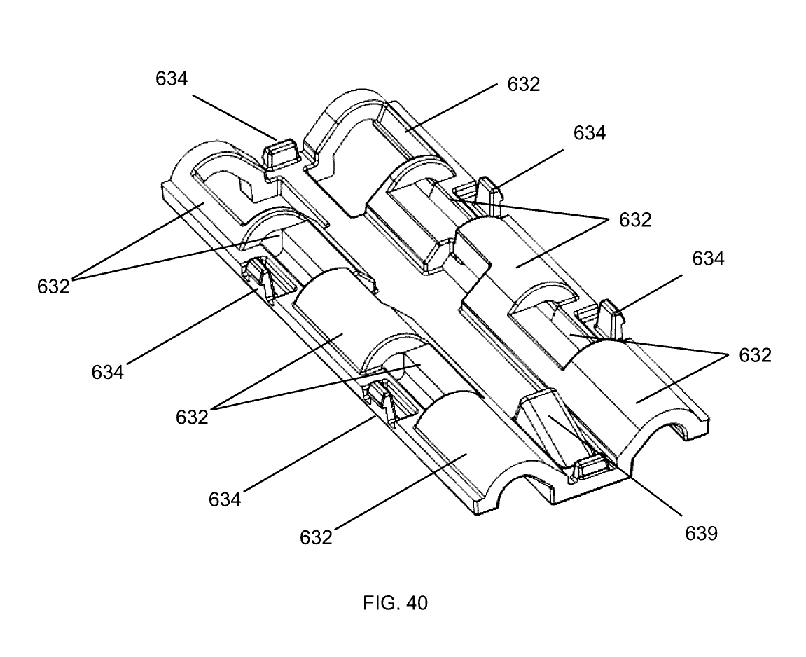

FIG. 40 is a perspective view of the base 630. The base has a rear end 631 and a front end 639. A needle aperture 633 is located at the front end 639. Clips 634 are present along the perimeter of the base, for engaging the casing 660. Knuckles 632 are also present on both sides of the base.

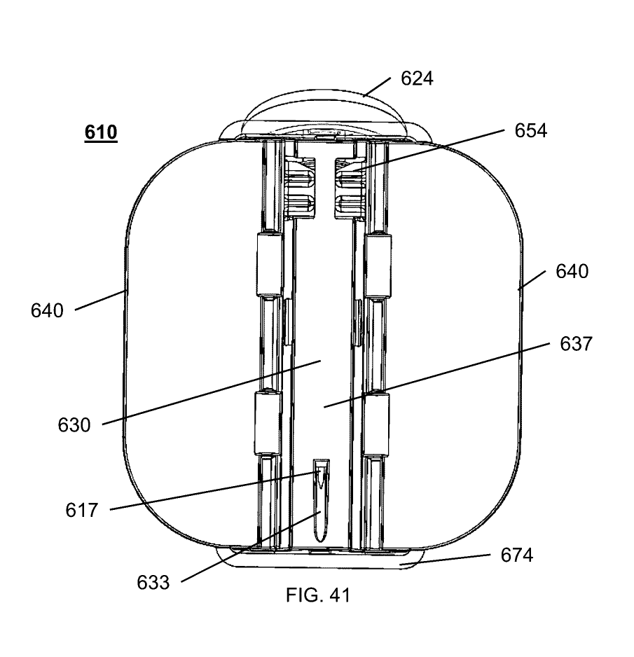

FIG. 41 is a bottom view of the device 610. The needle aperture 633 is shown, with needle 617 also illustrated. The front wall 674 of the casing is also visible. The wings 640 are on either side of the base 630. The actuators 654 are also visible near the rear of the device, along with the end cap 624. An antimicrobial material or a waterproof adhesive can be present on the exterior surface 637 of the base. The base can be made from a soft material, like silicone, or be covered by such a material to enhance grip and sealing as well.

Referring now to FIG. 33 and to FIG. 37, the slider track 662 is angled relative to the base, at an angle of about 10 degrees to about 30 degrees.

Referring now to FIG. 34 again, the firing mechanism is deployed by placing the base 630 against the surface of a patient's skin. While the base remains fixed against the patient's skin (e.g. by adhesion), the wings 640 are depressed downward towards the patient, as indicated by the arrows illustrated in FIG. 34. This will cause a tent of skin to be pulled upwards. This causes the actuators 654 to rotate around the hinge formed by the base 630 and wings 640, or out to the sides of the device, until the actuators no longer engage the stop wall 628 of the slider 620/retaining ring 622. The spring then extends, pushing the slider 620 to move along slider tracks 662 located on both sides of the casing 660, and causing the needle 617 to deploy through the needle aperture 633 in the base and be inserted into the skin of the patient. The needle 617 is still partially held within the device, at least at the needle hub 619, to maintain the general angle of injection and to allow slight vertical pivot to accommodate skin movement.

The needle 617, once injected into the dermis of a patient, may vary in position. However, the needle 617 is held firm within the device and at least the needle hub 619. Needle 617 position variance within aperture 633 may be due to expelled hydration fluid within a subcutaneous skin region pushing up against the needle. An oblong shape allows for needle 617 position variance without displacing needle 617 from the device or causing unnecessary patient discomfort or tissue trauma.

It is noted that in alternative embodiments, it is contemplated that the slider 620 and the actuators 654 can be shaped so that a spring is not needed to deploy the needle. Instead, the slider 620 and the actuator 654 are shaped so that the actuator maintains physical contact with the slider and pushes the slider 620 downwards as the actuator rotates around the hinge. This results in the slider 620 being deployed by mechanical movement, rather than energy stored in the spring.

When it is desired to remove the needle 617 from the skin, referring to FIG. 37, the user pulls back on the slider handles 626 of the slider 620, thereby moving the slider backwards along the slider tracks 662 and against the bias of the spring. To ensure that the slider 620 cannot be pushed back out after it has been retracted from the skin, the casing 660 includes recessed notches 664 that are generally located at the rear end of and below the slider tracks 662. The slider handles 626 can be pushed down into the recessed notches, once the slider 620 has been pulled backward a distance that is great enough to retract the needle 617 back into the casing 660. The recessed notches maintain the slider 620 in the retracted, undeployed position, thereby preventing accidental exposure of the needle 617. A mechanism is also contemplated where the slider 620 can be irreversibly locked into the retracted position for safety.

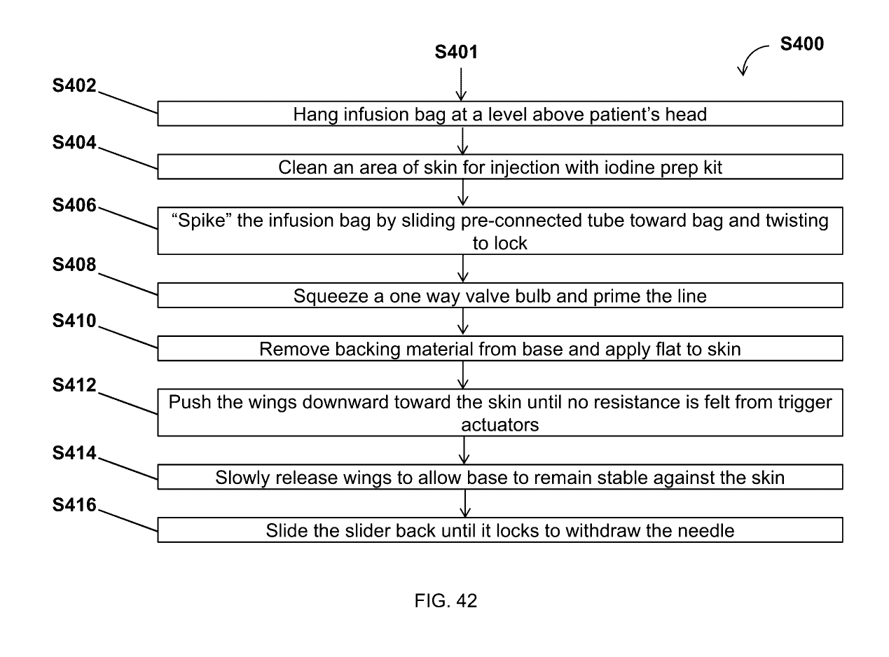

With reference to FIG. 42, an exemplary method S400 for performing subcutaneous rehydration begins at S401. Method S400 may be performed with deployment device 610 or other similar devices. At S402, an infusion bag is hung at a level above a patient's head. At S404, an area of skin is cleaned for injection with an iodine prep kit or the like. In one embodiment, the area of skin cleaned for injection is at least one of a back, thigh, or abdomen bodily area. At S406, the infusion bag is penetrated or “spiked” by sliding a pre-connected tube towards the bag and twisting to lock. At S408, a one way valve bulb is squeezed to prime the tube. It should be noted that S406 or S408 may be performed in the alternative or in combination to prime the tube. At S410, a rigid backing material is removed from the base and applied flat to the skin. This can help in preventing the wings from being folded downward during storage/shipment. At S412, push the wings downward toward the skin until no resistance is felt from trigger actuators. This action may pull up a tent of skin and automatically deploy a needle. At S414, wings are slowly released to allow base to remain stable against the skin. At S416, after fluids have been delivered to the patient, a slider is translated until locked in order to withdraw the needle from the patient.

It should be noted that any one of deployment devices 110, 210, 310, 410, 510, and 610 can be used as the deployment device for any one of the aforementioned systems 100, 400. It should also be noted that while deployment devices 110, 210, 310, 410, 510, 610 may be used as a secondary/temporizing means for rehydrating patients in the developing world with dehydrating diarrhea and emesis for whom 1) oral rehydration is either not possible (because of emesis, unresponsiveness, or young age) or not sufficient to offset fluid losses, and 2) access to clinically-trained personnel capable of administering intravenous fluids will be significantly delayed, there are also other significant applications. A device used in the third-world child rehydration setting may be the most basic, employing a smaller simple needle, and important further adaptions to the devices 110, 210, 310, 410, 510, 610 may necessarily be contemplated for providing subcutaneous hydration in different settings.

For example, the deployment device 110, 210, 310, 410, 510, 610 and/or systems 100, 400 may be adapted for otherwise extreme or military use on the battlefield where adults may face delays in receiving fluid replacement/resuscitation. Deviations from described devices may include using materials which can withstand excessive environmental temperatures for long periods of time and may be either wrapped in Ultraviolet (UV)-protective materials or manufactured from materials which degrade minimally in ultraviolet light. In order to avoid excessive solar heating of fluids in outdoor environments, the infusion bag 102, 402 may be light in color and nontransparent with the exception of a small, clear window for assessing turbidity of the fluid. The crystalloid fluids within infusion bag 102, 402 may also contain appropriately-dosed recombinant hyaluronidase, an enzyme which reversibly lowers the resistance to infusion of fluids into the subcutaneous space to allow more rapid infusion. The devices 110, 210, 310, 410, 510, 610 may also include a larger bore needle. The devices may be manufactured such that a second or subsequent bag can be attached at entry point when the first infusion bag 102, 402 has been emptied.

Another adaption of the devices 110, 210, 310, 410, 510, 610 may be for non-acute, developing world applications, e.g., the care of patients who require maintenance fluids in an institutional or home setting such as nursing home patients, hospice patients, and home care patients, patients who will have surgery the following day and who are allowed nothing by mouth). Deviations from the described devices 110, 210, 310, 410, 510, 610 may include, in addition to an antibacterial preparation, placing an adhesive covering the portion of the base and/or wings that would also be impregnated with an emulsion of prilocaine and lidocaine local anesthetic. The same procedure for activating devices 410, 510 may be followed except a user would wait five minutes between application of the wings and pinch deployment of the needle. Additionally, an injection port may be included in the infusion bag 102, 402 for adding medications.

The present disclosure has been described with reference to exemplary embodiments. Obviously, modifications and alterations will occur to others upon reading and understanding the preceding detailed description. It is intended that the present disclosure be construed as including all such modifications and alterations insofar as they come within the scope of the appended claims or the equivalents thereof.

1. A deployment device (610) for subcutaneous hydration, comprising:
a base (630) including a needle aperture (633);
a casing (660) attached to the base;
a firing mechanism (650) within the casing (660) for deploying a needle (617) from the needle aperture (633); and
a wing (640) attached to the base, the wing including an actuator (654) that regulates the firing mechanism (650);
wherein the firing mechanism deploys the needle (617) through the aperture (633) when the wing (640) is moved so that the actuator (654) triggers the firing mechanism (650).

2. The deployment device (610) of claim 1, wherein the firing mechanism (650) includes:
a slider (620) with handles (626) that travel through a slider track (662) within the casing (660);
a stop wall (628) that engages the actuator (654); and
the needle (617).

3. The deployment device (610) of claim 2, wherein the stop wall (628) is part of a retaining ring (622) that is adapted to fix the needle (617) in place on the slider (620).

4. The deployment device (610) of claim 2, wherein the slider (620) includes a hollow passage (629), such that fluid can pass from a rear portion (621) of the slider through the slider to the needle (617).

5. The deployment device (610) of claim 2, wherein the firing mechanism (650) includes a spring between the casing (660) and a rear portion (621) of the slider.

6. The deployment device (610) of claim 1, wherein the casing (660) includes a slider track (662) on two opposite sides, the slider track being angled relative to the base (630).

7. The deployment device (610) of claim 6, wherein the casing (660) further includes at least one recessed notch (664) located below the slider track (662) for maintaining the firing mechanism (650) in a retracted position.

8. The deployment device (610) of claim 1, wherein the casing (660) and the base (630) define a barrel (616), and the device further comprises an end cap (624) that engages a rear wall of the casing (660) to enclose the barrel (516).

9. The deployment device (610) of claim 8, wherein the firing mechanism (650) is inserted into the barrel (616) and is completely separable from the casing (660) and the base (630).

10. The deployment device (610) of claim 11, wherein the end cap (624) further includes projecting walls (636) that define a rear end of a slider track (662).

11. The deployment device (610) of claim 1, wherein the base (630) includes knuckles (632), and the wing (640) includes knuckles (642), such that the wing and the base are hinged together.

12. The deployment device (610) of claim 1, wherein the wing (640) comprises a frame (644), and the actuator (654) extends transversely to the frame.

13. The deployment device (610) of claim 1, wherein the needle (617) is deployed from the base at an angle.

14. The deployment device (610) of claim 11, wherein the angle is from about 10 degrees to about 30 degrees.

15. The deployment device of claim 1, wherein the aperture (633) accommodates movement of the needle (617) when deployed.

16. The deployment device (610) of claim 1, wherein an exterior surface of the base (630) includes at least an antimicrobial material or a waterproof adhesive.

17. A system (100, 400) for providing subcutaneous hydration, comprising:
an infusion bag (102, 402) including a hydration fluid;
a tubing (108, 408) removably attached to the infusion bag (102, 402) an at least partially encompassed by a valve (106, 406);
a deployment device (610) according to claim 1 including a needle (617);
wherein the deployment device (610) is attached to the tubing (108, 408) and is operative to provide subcutaneous hydration through the needle (617).

18. A method for performing subcutaneous hydration, comprising:
providing a deployment device (610) of claim 1;
hanging an infusion bag (102, 104) at a level above a patient's head;
cleaning an area of skin of the patient;
penetrating the infusion bag (102, 104) by sliding a pre-connected tube towards the bag and twisting to lock;
squeezing a one-way valve bulb to prime the tube;
removing a backing material from a base (630) of the deployment device (610) and applying the base flat to the skin of the patient;
pushing at least one wing (640) of the deployment device (610) downward toward the skin of the patient until a needle (617) is automatically deployed; and,
sliding a slider (640) until locked in order to withdraw the needle from the patient.
